(19) United States
(12) Patent Application Publication
(10) Pub. No.: US 2025/0082696 A1
(43) Pub. Date: Mar. 13, 2025

(54) METHOD FOR PROVIDING RELIEF FROM FRUCTAN OR FRUCTOSE INDUCED ABDOMINAL DISCOMFORT

(71) Applicant: Novozymes A/S, Bagsvaerd (DK)

(72) Inventors: Kristian Bertel Romer M. KROGH, Bagsvaerd (DK); Jeffrey SCHULTCHEN, Berlin (DE); Lea Benedicte Skov HANSEN, Lyngby (DK); Sameer KULKARNI, Lyngby (DK); Jason QUINLAN, Woodland, CA (US)

(73) Assignee: Novozymes A/S, Bagsvaerd (DK)

(21) Appl. No.: 18/726,653

(22) PCT Filed: Jan. 6, 2023
(86) PCT No.: PCT/EP2023/050216

§ 371 Date: Jul. 3, 2024

(51) Int. Cl. A61K 35/742; A61K 38/47

(52) U.S. Cl. CPC A61K 35/742; A61K 38/47; C12Y 302/01007; C12Y 302/01026; C12Y 302/01065; C12Y 302/0108

(57) ABSTRACT

The present invention relates to a composition comprising one or more fructanases and one or more Lactobacillus strains to provide relief from or prevent fructan and/or fructose induced abdominal discomfort; digest fructan and/or fructose or improve digestion of fructan and/or fructose; and/or provide digestive comfort after digestion of fructan and/or fructose on consumption of products containing fructan and/or fructose to a subject. The present invention also relates a Lactobacillus strain or a fructanse for providing relief from fructan and/or fructose induced abdominal discomfort to a subject, wherein the Lactobacillus strain in presence of one or more fructanases results in at least 20-50% reduction of gas pressure development, when tested against the fecal fermentation of the fiber alone across different donors.

### REFERENCE TO SEQUENCE LISTING

This application contains a Sequence Listing in computer readable form. The computer readable form is incorporated herein by reference.

### REFERENCE TO A DEPOSIT OF BIOLOGICAL MATERIAL

This application contains a reference to a deposit of biological material, which deposit is incorporated herein by reference. For complete information see last paragraph of the description.

### FIELD OF THE INVENTION

The present invention relates to a method to reduce fructans and fructose in the gastrointestinal (GI) tract thus reducing fructan or fructose induced abdominal discomfort.

### BACKGROUND

Fructans are oligomers and polymers of fructose molecules of varying degree. Fructans such as Inulin fibers are widely distributed in many types of fruits and vegetables such as bananas, onions, garlic, artichokes etc. and in widely consumed cereals such as wheat, rye and barley. Fructans belong to a class of fermentable fibers called FODMAPS (fermentable oligo-, di-, monosaccharides and polyols). FODMAPS have been implicated in causing abdominal discomfort such as gas, bloating, diarrhea, constipation, stomach pain in sensitive population. Humans do not have the fructan-digesting enzyme, thus fructans cannot be effectively digested in the small intestine like other nutrients and are fermented instead in the human gut by the gut microbiome.

A fructan rich diet is consumed in many parts of the world which includes a diet which is rich in wheat, rye, barley etc. Many people experience symptoms of bloating, gas, diarrhea, constipation, and/or abdominal discomfort following the hours after eating fructan-containing food. Although several molecules are implicated in causing abdominal discomfort the role of these molecules remain unclear, more recent studies indicate towards fructans as more likely culprits to induce symptoms in patients who report sensitivity to a diet rich in fructans such as a wheat-based diet. As a general practice, the avoidance of fructan containing diet such as e.g., a wheat containing meal is pursued to reduce the symptoms and discomfort. However, this may be difficult because of the large number of food products that contain fibers such as fructans and that include food made up of wheat such as e.g., bread, pasta, cereals, couscous, cakes, pastries, biscuits, doughnuts, hydrolyzed vegetable protein (HVP), beer and soy sauce etc. excluding fructan containing foods such as wheat from the diet is not only challenging but also negatively impacts the quality of life. Currently, there are several products which are targeted towards gluten degradation, but none towards targeting fructans.

WO 2020/169545 A1 relates to a method for reducing fructan in a fructan-containing food product by adding an invertase belonging to enzyme classification EC 3.2.1.26 to the food product.

US2008187525 AA relates to enhancing the in vivo colonization of probiotic microorganisms that include digestive enzymes and probiotic microorganisms, and polysorbate surfactants.

There is still a need for effective methods of reducing discomfort from foods containing fructans.

### SUMMARY OF THE CLAIMED INVENTION

The present invention relates to a composition comprising one or more fructanases and one or more Lactobacillus strains for a use which is selected from a group consisting of providing relief from or preventing fructan and/or fructose induced abdominal discomfort; digesting fructan and/or fructose or improving digestion of fructan and/or fructose; and/or providing digestive comfort after digestion of fructan and/or fructose on consumption of products containing fructan and/or fructose to a subject. In one aspect, the fructanase belongs to the GH32 family. In another or further aspect, the Lactobacillus strain is Lactobacillus johnsonii or Lactobacillus crispatus.

The present invention further relates to a Lactobacillus strain for providing relief from fructan and/or fructose induced abdominal discomfort to the subject, wherein the Lactobacillus strain in presence of one or more fructanases results in at least 20% reduction of gas pressure development, when tested against the fecal fermentation of the fiber alone across different donors.

The present invention further relates to a fructanase for providing relief from fructan and/or fructose induced abdominal discomfort to a subject, wherein the fructanase results in at least 20% reduction of gas pressure development, when tested against the fecal fermentation of the fiber alone across five different donors.

Also, a composition comprising one or more fructanases and one or more Lactobacillus strains is covered by the invention.

### OVERVIEW OF SEQUENCE LISTING

SEQ ID NO: 1 is the mature amino acid sequence of exo-inulinase from Fructozyme L™ from Novozymes A/S, Denmark.

SEQ ID NO: 2 is the mature amino acid sequence of endo-inulinase from Fructozyme L™ from Novozymes A/S, Denmark.

SEQ ID NO: 3 is the mature amino acid sequence of invertase from Fructozyme L™ from Novozymes A/S, Denmark.

### BRIEF DESCRIPTION OF THE DRAWINGS

A more complete understanding of the present invention may be derived by referring to the detailed description and claims when considered in connection with the Figures.

FIG. 1A shows the growth curves of Lactobacillus strain HH15 in presence of glucose, fructose, fructo-oligo saccharide (FOS) or inulin in 1:2 diluted standard medium at OD600 over 20 hours.

FIG. 1B shows the growth curves of Lactobacillus strain HH15 in presence of glucose, fructose, fructo-oligo saccharide (FOS), saccharose, raffinose, stachyose or galactose in 1:2 diluted standard medium at OD600 over 20 hours.

FIG. 2 shows the growth of Lactobacillus strain HH15 as difference between the start and end values at OD600 in presence of glucose, fructose, fructo-oligo saccharide (FOS), saccharose, raffinose, stachyose or galactose in 1:2 diluted standard medium at OD600 over 20.

FIG. 3A shows the effect of Enzyme A alone, Lactobacillus strain HH15 alone, and the effect of the combination of Enzyme A and Lactobacillus strain HH15 together on fructan (substrate) induced gas pressure in a fecal fermentation experiment on donor set 1.

FIG. 3B shows the same graph as FIG. 3A with one outlier excluded from donor set 1 and a corrected donor set 2

FIG. 4 (4A, 4B, 4C and 4D) shows the effect of either Enzyme A, Enzyme B, Enzyme C or Enzyme D each alone, Lactobacillus strain HH15 alone, and the effect of the combination of either Enzyme A, Enzyme B, Enzyme C or Enzyme D and Lactobacillus strain HH15 together on fructan (substrate) induced gas pressure in a fecal fermentation experiment on a donor set 3.

FIG. 5 (5A, 5B, and 5C) shows the effect of either Enzyme A, Enzyme B or Enzyme E each alone, Lactobacillus strain HH15 alone, and the effect of the combination of either Enzyme A, Enzyme B or Enzyme E and Lactobacillus strain HH15 together on fructan (substrate) induced gas pressure in a fecal fermentation experiment a donor set 4.

FIG. 6 (6A and 6B) shows the effect of either Enzyme A or Enzyme F each alone, Lactobacillus strain HH15 alone, and the effect of the combination of either Enzyme A or Enzyme F and Lactobacillus strain HH15 together on fructan (substrate) induced gas pressure in a fecal fermentation experiment on a donor set 5.

FIG. 7 (7A and 7B) shows the effect of enzyme Enzyme A alone, Lactobacillus strain HH15 alone, and the effect of the combination of Enzyme A and Lactobacillus strain HH15 together on fructan inulin or fructan FOS (substrate) induced gas pressure in a fecal fermentation experiment on a donor set 6. For more details see Example 3, and Example 6.

FIG. 8 shows the growth of Lactobacillus strain HH22 as difference between the start and end values at OD600 in presence of glucose, fructose and inulin in 1:2 diluted standard medium at OD600 over 20 hours.

FIG. 9 shows the effect of enzyme Enzyme A alone, Lactobacillus strain HH22 alone, and the effect of the combination of Enzyme A and Lactobacillus strain HH22 together on fructan (substrate) induced gas pressure in a fecal fermentation experiment on a fecal donor set 7.

FIG. 10 shows the effect of enzyme Enzyme A alone, Lactobacillus strain HH15 alone, and the effect of the combination of Enzyme A and Lactobacillus strain HH15 together on fructan (substrate) induced gas pressure in a fecal fermentation experiment on a fecal donor set 8. For more details see Example 3, Example 4, and Example 5.

FIG. 11A shows a bar plot with the average relative abundance of the probiotics strain HH15 on the y-axis in percentage of the total microbiome.

FIG. 11B shows a bar plot with the average relative abundance of the genus Bifidobacterium on the y-axis in percentage of the total microbiome.

### DEFINITIONS

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the term “flatulence” is defined as the presence of excessive amounts of gas in the stomach or intestine, which may result in the expulsion of the gas through the anus.

As used herein, the term “abdominal discomfort” is an expression for discomfort felt in the abdomen such as the feeling of pain, ache, cramp, nausea, irritation, bloating or the like in the abdomen or any other symptoms associated with fructan and/or fructose intolerance, fructanase deficiency and/or digesting fructan and/or fructose.

As used herein, the term “digest” means break down or help break down food in the gastrointestinal tract into substances that can be absorbed and used by the body. For purposes of the present invention, the term “digesting fructan and/or fructose” is breaking down of fructan and/or fructose in the gastrointestinal tract. For purposes of the present invention, the term “improving digestion of fructan and/or fructose” is digesting fructan and/or fructose for subjects who have difficulty in digesting fructan and/or fructose.

As used herein, the term “digestive comfort” in the present invention means absorption of fructan and/or fructose from gastrointestinal tract without causing any abdominal discomfort.

As used herein, the term “diarrhea” is a condition of having three or more loose or liquid stools per day, or as having more stools than is normal for that person. Acute diarrhea is defined as an abnormally frequent discharge of semisolid or fluid fecal matter from the bowel, lasting less than 14 days.

As used herein, the term “constipation” refers to infrequent bowel movements where symptoms may include hard stools, straining with bowel movements, excessive time needed to pass a bowel movement, pain with bowel movements secondary to straining, abdominal pain, abdominal bloating and/or the sensation of incomplete bowel evacuation.

As used herein, the term “abdominal bloating” is an expression for gas in abdomen that leads to a sensation that the abdomen is full or distended.

As used herein, the term “fructans” are oligomers and polymers of fructose molecules. Fructans such as inulin fibers are e.g., widely distributed in many types of fruits and vegetables such as bananas, onions, garlic, artichokes etc. and in widely consumed cereals such as e.g., wheat, rye, barley etc. They belong to a class of fermentable fibers called FODMAPs (fermentable oligo, di, monocaccharides and polyols).

As used herein, the term “fructanase” may also be termed fructan hydrolase and is the term for any enzyme that catalyzes the hydrolysis of fructans such as inulin, levan, and/or sucrose. Examples of fructanases include without limitation: endo-inulinase (EC 3.2.1.7), exo-inulinase (EC 3.2.1.80), beta-fructofuranosidase, invert-ase (EC 3.2.1.26) and levanase (EC 3.2.1.65).

As used herein, the term “GH35 Family” refers to glycoside hydrolase family 32 and comprises the enzymes β-fructofuranosidases (EC 3.2.1.26), also called invertases, that hydrolyse terminal non-reducing β-D-fructofuranoside residues in β-D-fructofuranosides, enzymes that hydrolyze fructose containing polysaccharides such as endo-inulinases (EC 3.2.1.7) and exo-inulinases (EC 3.2.1.80), levanases (EC 3.2.1.65) and β-2,6-fructan 6-levanbiohydrolases (EC 3.2.1.64), fructan β-(2,1)-fructosidase/1-exohydrolase (EC 3.2.1.153) or fructan β-(2,6)-fructosidase/6-exohydrolases (EC 3.2.1.154), as well as enzymes displaying transglycosylating activities such as sucrose:sucrose 1-fructosyltransferases (EC 2.4.1.99), fructan:fructan 1-fructosyltransferase (EC 2.4.1.100), sucrose:fructan 6-fructosyltransferase (EC 2.4.1.10), fructan:fructan 6G-fructosyltransferase (EC 2.4.1.243) and levan fructosyltransferases (EC 2.4.1.-), sucrose:sucrose 6-fructosyltransferase (6-SST) (EC 2.4.1.-); cycloinulo-oligosaccharide fructanotransferase (EC 2.4.1.-) As used herein, the term “exo-inulinase” is the same as fructan β-fructosidase and comprises enzymes of the EC class EC 3.2.1.80 that hydrolyze fructose containing polysaccharides such as inulin and levan by hydrolyzing the terminal, non-reducing (2→1)- and (2→6)-linked β-D-fructofuranose residues in fructans. Exo-inulinase may also hydrolyze sucrose. Other names used for exo-inulinases include but are not limited to exo-β-D-fructosidase; exo-β-fructosidase; polysaccharide β-fructofuranosidase; fructan exohydrolase and β-D-fructan fructohydrolase.

As used herein, the term “endo-inulinase” comprises enzymes of the EC class EC 3.2.1.7 that hydrolyze fructose containing polysaccharides by endohydrolyzing (2→1)-β-D-fructosidic linkages in fructose. Other names used for endo-inulinases include but are not limited to inulinase; inulase; indoinulinase; and 2,1-β-D-fructan fructanohydrolase.

As used herein, the term “invertase” is the same as β-fructofuranosidase and comprises enzymes of the EC class EC 3.2.1.26 that hydrolyse terminal non-reducing β-D-fructofuranoside residues in β-D-fructofuranosides. Invertase may e.g., use sucrose as substrate and may also catalyse fructotrans-ferase reactions. Other names used for invertase include but is not limited to saccharase; glucosucrase; β-h-fructosidase; β-fructosidase; invertin; sucrase; maxinvert L 1000; fructosylinvertase; alkaline invertase and acid invertase.

As used herein, the term “mature polypeptide” means a polypeptide in its mature form following N terminal and/or C-terminal processing (e.g., removal of signal peptide).

As used herein, “gas pressure measurement system” is a system designed to measure gas pressure where closed vessels equipped with a sensor are used for continuously measuring gas pressure within an observation period. The closed vessels may be e.g. Hungate tubes or 5 ml vials with a rubber septum (originally for cultivation of anaerobic microorganisms). For the purposes of the present invention, the gas pressure measurement system is used to measure the amount of gas generated in fecal samples with and without Lactobacillus strains and fructanases.

As used herein, “delta OD600” is a reference to a spectrometer method that is used to help estimate the concentration or “number of cells per volume” of bacteria or other cells within a liquid sample, where OD 600 is reference to the 600-nanometer (nm) wavelength used to measure optical density of the liquid sample. For purposes of the present invention,

delta OD 600=(final OD600 with fructose as the main carbon source in a sample)−(final OD600 without fructose in a sample).

As used herein, “sequence identity” is the relatedness between two amino acid sequences or between two nucleotide sequences. is described by the parameter “sequence identity”. For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

While certain embodiments of the present disclosure will hereinafter be described with reference to embodiments thereof, it will be understood by those of ordinary skill in the art that various changes in form and details may be made therein without departing from the spirit and scope of the present invention as defined by the claims.

### DETAILED DESCRIPTION OF THE INVENTION

The present invention relates to uses, composition and methods for providing relief from or preventing fructan and/or fructose induced abdominal discomfort, digesting fructan and/or fructose or improving digestion of fructan and/or fructose, and/or providing digestive comfort after digestion of fructan and/or fructose on consumption of products containing fructan and/or fructose to a subject.

The inventors of the present invention have surprisingly found that by applying one or more fructanases and/or Lactobacillus strains to a human, abdominal discomfort is reduced and/or relief is provided from the feeling of discomfort from e.g., diarrhea, pain, flatulence, nausea, constipation, bloating induced from fructan and/or fructose ingestion, flatulence and/or gas induced from fructan and/or fructose ingestion. In one aspect, fructan and/or fructose induced flatulence, gas formation and/or gas pressure creation in a human is reduced by the use, method, fructanase(s), Lactobacillus strain(s) or composition. In a further or alternative aspect, the invention is for a human sensitive to fructan and/or fructose rich foods.

In one embodiment of the invention, one or more Lactobacillus strains are provided to a human in presence of one or more fructanases, wherein the Lactobacillus strain is Lactobacillus johnsonii or Lactobacillus crispatus. The inventors surprisingly identified the power of instant fructan break down into fructoses by fructanases and fructose digestion by Lactobacillus strains provides relief from fructan and/or fructose induced abdominal discomfort to subject. This dual action concept may be used to efficiently tackle fructan and/or fructose induced gastrointestinal symptoms by combining the fructanase with the Lactobacillus strains in a dosage form. One embodiment of the present invention thus covers a dosage form comprising one or more Lactobacillus strains and one or more fructanases. In an embodiment, the Lactobacillus strain is Lactobacillus johnsonii or Lactobacillus crispatus. On consumption of the dosage form with or before consuming the fructan and/or fructose containing meal, the fructanase immediately hydrolyses dietary fructan to fructose while the Lactobacillus strains of the present invention on the other hand causes fructose digestion. Thus, the mode-of-action of the combination delivers protection to consumers from dietary fructan and/or fructose induced gastrointestinal symptoms and manages fructan and/or fructose intolerance effectively improving the quality of life. The present invention provides relief from fructan and/or fructose induced abdominal discomfort in humans affected by and/or intolerant to fructan and/or fructose, having difficulty in digesting fructan and/or fructose.

### Lactobacillus Strains

The one or more Lactobacillus strains of the invention or for use in the present invention may be any Lactobacillus strains that digest fructose. The one or more Lactobacillus strains may e.g. be Lactobacillus johnsonii or Lactobacillus crispatus. The level of digestion may e.g., be determined by measuring the maximum growth rate of the Lactobacillus strains on a medium containing fructose and/or by measuring the delta OD600 value in the medium containing fructose according to Example 2.

In one embodiment, the Lactobacillus strains grow on fructose in vitro and/or in the gastrointestinal tract of humans. In one embodiment, the Lactobacillus strains according to the invention or for use in the invention reduce gas production of fecal matter induced with fructan and/or fructose in a gas pressure measurement system. In an embodiment, the Lactobacillus strains in presence of one or more fructanases reduce at least 20-50% gas pressure development, when tested against the fecal fermentation of the fiber alone across different donors. In another embodiment, the Lactobacillus strains in presence of one or more fructanases reduce up to 50% gas pressure development, when tested against the fecal fermentation of the fiber alone across different donors. In a preferred embodiment, the Lactobacillus strains in presence of one or more fructanases reduce at least 20%, such as at least 40% or least 50% gas pressure development, when tested against the fecal fermentation of the fiber alone across different donors.

In an embodiment of the present invention, the one or more Lactobacillus strains of the present invention and for use, for a method and for a composition of the present invention is Lactobacillus johnsonii strain HH15 deposited under deposit accession number DSM 33901 or Lactobacillus crispatus strain HH22 deposited under deposit accession number DSM 34452. In another embodiment of the present invention, the Lactobacillus johnsonii strain is Lactobacillus johnsonii strain having all the identifying characteristics of Lactobacillus johnsonii strain having deposit number DSM 33901, or a mutant of Lactobacillus johnsonii strain deposited under deposit accession number DSM 33901. In another embodiment of the present invention, the Lactobacillus crispatus strain is Lactobacillus crispatus strain having all the identifying characteristics of Lactobacillus crispatus strain having deposit number DSM 34452, or a mutant of Lactobacillus crispatus strain deposited under deposit accession number DSM 34452.

In another embodiment, the one or more Lactobacillus strains breaks down galactan and/or galactose. In yet another embodiment,

### Fructanases

The one or more fructanases of the invention and for the use, method or composition of the invention may be any fructanases that reduce the gas pressure in a gas pressure tests as described in the examples of the patent application. In one aspect, the fructanases are selected from the fructanases of the glycoside hydrolase family GH32. In an embodiment of the present invention, the one or more fructanases are endo-inulinase (EC 3.2.1.7), exo-inulinase (EC 3.2.1.80), beta-fructofuranosidase, invertase (EC 3.2.1.26) and/or levanase (EC 3.2.1.65). In another embodiment, the one or more fructanases are invertase (EC 3.2.1.26), endo-inulinase (EC 3.2.1.7) and/or exo-inulinase (EC 3.2.1.80). In another embodiment, the one or more fructanases are endo-inulinase (EC 3.2.1.7) and/or exo-inulinase (EC 3.2.1.80).

In another embodiment, the one or more fructanases breaks down fructan to fructose. In an embodiment, the fructan is fructo-oligo saccharides (FOS). In yet another embodiment, the one or more fructanases induces the growth of Bifidobacterium during hydrolysis of inulin.

a. Exo-Inulinase

In an embodiment, the exo-inulinase is a polypeptide selected from the group consisting of:
(a) a polypeptide having at least 80% sequence identity to SEQ ID NO:1;
(b) a polypeptide derived from SEQ ID NO:1 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
(c) a polypeptide derived from the polypeptide of (a) or (b), wherein the N- and/or C-terminal end has been extended by addition of one or more amino acids, and
(d) a fragment of the polypeptide of (a), (b) or (c).

In another embodiment, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide sequence of SEQ ID NO:1.

In a preferred embodiment of the present invention, the exo-inulinase is a polypeptide comprising, consisting essentially of, or consisting of SEQ ID NO:1.

In an embodiment, the polypeptide may have an N-terminal and/or C-terminal extension of one or more amino acids, e.g., 1-5 amino acids.

In one embodiment, the polypeptide is derived from SEQ ID NO:1 by substitution, deletion or addition of one or several amino acids. In some embodiments, the polypeptide is a variant of SEQ ID NO:1 comprising a substitution, deletion, and/or insertion at one or more positions. In one embodiment, the number of amino acid substitutions, deletions and/or insertions introduced into the polypeptide of SEQ ID NO:1 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

b. Endo-Inulinase

In an embodiment, the endo-inulinase is a polypeptide selected from the group consisting of:
(a) a polypeptide having at least 80% sequence identity to SEQ ID NO:2;
(b) a polypeptide derived from SEQ ID NO:2 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
(c) a polypeptide derived from the polypeptide of (a) or (b), wherein the N- and/or C-terminal end has been extended by addition of one or more amino acids, and
(d) a fragment of the polypeptide of (a), (b) or (c).

In another embodiment, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide sequence of SEQ ID NO:2.

In another preferred embodiment of the present invention, the endo-inulinase is a polypeptide comprising, consisting essentially of, or consisting of SEQ ID NO:2.

In an embodiment, the polypeptide may have an N-terminal and/or C-terminal extension of one or more amino acids, e.g., 1-5 amino acids.

In one embodiment, the polypeptide is derived from SEQ ID NO:2 by substitution, deletion or addition of one or several amino acids. In some embodiments, the polypeptide is a variant of SEQ ID NO:2 comprising a substitution, deletion, and/or insertion at one or more positions. In one embodiment, the number of amino acid substitutions, deletions and/or insertions introduced into the polypeptide of SEQ ID NO:2 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

c. Invertase

In an embodiment, the invertase is a polypeptide selected from the group consisting of:
(a) a polypeptide having at least 80% sequence identity to SEQ ID NO:3;
(b) a polypeptide derived from SEQ ID NO:3 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
(c) a polypeptide derived from the polypeptide of (a) or (b), wherein the N- and/or C-terminal end has been extended by addition of one or more amino acids, and
(d) a fragment of the polypeptide of (a), (b) or (c).

In another embodiment, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide sequence of SEQ ID NO:3.

In another preferred embodiment of the present invention, the invertase is a polypeptide comprising, consisting essentially of, or consisting of SEQ ID NO:3.

In an embodiment, the polypeptide may have an N-terminal and/or C-terminal extension of one or more amino acids, e.g., 1-5 amino acids.

In one embodiment, the polypeptide is derived from SEQ ID NO:3 by substitution, deletion or addition of one or several amino acids. In some embodiments, the polypeptide is a variant of SEQ ID NO:3 comprising a substitution, deletion, and/or insertion at one or more positions. In one embodiment, the number of amino acid substitutions, deletions and/or insertions introduced into the polypeptide of SEQ ID NO:3 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

### Composition

In one embodiment, the present invention relates to a composition for providing relief from or preventing fructan and/or fructose induced abdominal discomfort; digesting fructan and/or fructose or improving digestion of fructan and/or fructose; and/or providing digestive comfort after digestion of fructan and/or fructose on consumption of products containing fructan and/or fructose; to a subject, wherein the composition comprises one or more Lactobacillus strains and one or more fructanases. In an embodiment, the Lactobacillus strain is Lactobacillus johnsonii or Lactobacillus crispatus. In an embodiment, the subject is a human. In an embodiment of the composition, the dosage form of the composition can be a single dosage form or multiple dosage form. In an embodiment of the composition, the composition can be in a dosage form such as, but not limited to, capsules, powders, microcapsules, tablets, dragees, pellets granules, chewables and liquid forms. In an embodiment of the composition, the composition comprises from 1E+08 Colony Forming Units (CFU) to 1E+11 CFU of the one or more Lactobacillus strains. In a preferred embodiment, the composition comprises about 1E+09 CFU of the one or more Lactobacillus strains. In an embodiment of the composition, the composition comprises 1 to 5 milligram (mg) enzyme protein, preferably 1.5 to 4.5 mg enzyme protein, preferably 2 to 4 mg enzyme protein, preferably 2.5 to 3.5 mg enzyme protein, preferably 2 to 3 mg enzyme protein of the one or more fructanases. In a preferred embodiment, the composition comprises about 1.5 to 2.5 mg enzyme protein of the one or more fructanases. In another embodiment, the composition comprises 3 to 5 mg enzyme protein, preferably 3.2 to 4.5 mg enzyme protein 1.5 to 2.5 mg enzyme protein of the one or more endo-inulinase. In another embodiment, the composition comprises 1.5 to 2 mg enzyme protein, preferably 1.3 to 1.9 mg enzyme protein of the one or more exo-inulinase.

In an embodiment where the composition of the invention comprises one or more fructanases, the fructanase(s) are immediately available for fructan breakdown in the gastrointestinal tract. In an embodiment of the composition, the one or more fructnases of the composition break down fructan to fructose that the subject consumes after consumption of the composition and the one or more Lactobacillus strains break down fructose. The composition may be provided to the subject 1, 2 or 3 times in a day to provide relief from or prevent fructan and/or fructose induced abdominal discomfort; digest fructan and/or fructose or improve digestion of fructan and/or fructose; and/or provide digestive comfort after digestion of fructan and/or fructose on consumption of products containing fructan and/or fructoseto a subject. In one embodiment, the composition is provided to the subject two times per day to provide relief from or prevent fructan and/or fructose induced abdominal discomfort; digest fructan and/or fructose or improve digestion of fructan and/or fructose; and/or provide digestive comfort after digestion of fructan and/or fructose on consumption of products containing fructan and/or fructose; to a subject. In a preferred embodiment, the composition may be provided to the subject once in a day to provide relief from or prevent fructan and/or fructose induced abdominal discomfort; digest fructan and/or fructose or improve digestion of fructan and/or fructose; and provide digestive comfort after digestion of fructan and/or fructose on consumption of products containing fructan and/or fructose to a subject.

### Uses and Methods

Also disclosed herein is the use of one or more Lactobacillus strains or a composition comprising such, for a use which is selected from a group consisting of:
a. providing relief from or preventing fructan and/or fructose induced abdominal discomfort to a subject,
b. digesting fructan and/or fructose or improving digestion of fructan and/or fructose for a subject;
c. providing digestive comfort after digestion of fructan and/or fructose on consumption of products containing fructan and/or fructose to a subject; and
d. any combination thereof.

In an embodiment, the Lactobacillus strain is Lactobacillus johnsonii or Lactobacillus crispatus. In one embodiment, the use is for providing relief from or preventing fructan and/or fructose induced abdominal discomfort to a subject. In one embodiment, the use is for digesting fructan and/or fructose or improving digestion of fructan and/or fructose for a subject. In one embodiment, the use is for providing digestive comfort after digestion of fructan and/or fructose on consumption of products containing fructan and/or fructose to a subject. In an embodiment, the subject is a human.

Also disclosed herein is the use of one or more fructanases or a composition comprising such, for a use which is selected from a group consisting of:
a. providing relief from or preventing fructan and/or fructose induced abdominal discomfort to a subject,
b. digesting fructan and/or fructose or improving digestion of fructan and/or fructose for a subject;
c. providing digestive comfort after digestion of fructan and/or fructose on consumption of products containing fructan and/or fructose to a subject; and
d. any combination thereof.

In one embodiment, the use is for providing relief from or preventing fructan and/or fructose induced abdominal discomfort to a subject. In one embodiment, the use is for digesting fructan and/or fructose or improving digestion of fructan and/or fructose for a subject. In one embodiment, the use is for providing digestive comfort after digestion of fructan and/or fructose on consumption of products containing fructan and/or fructose to a subject. In an embodiment, the subject is a human.

In another embodiment, the present invention relates to a method for providing relief from or preventing fructan and/or fructose induced abdominal discomfort; digesting fructan and/or fructose or improving digestion of fructan and/or fructose; and/or providing digestive comfort after digestion of fructan and/or fructose on consumption of products containing fructan and/or fructose to a subject; the method comprising providing to a subject in need thereof an effective amount of a composition comprising one or more fructanases and one or more Lactobacillus strains. In an embodiment, the Lactobacillus strain is Lactobacillus johnsonii or Lactobacillus crispatus.

In one embodiment, the method is for providing relief from or preventing fructan and/or fructose induced abdominal discomfort to a subject. In one embodiment, the method is for digesting fructan and/or fructose or improving digestion of fructan and/or fructose for a subject. In one embodiment, the method is for providing digestive comfort after digestion of fructan and/or fructose on consumption of products containing fructan and/or fructose to a subject.

In an embodiment of the method, the composition is provided 1, 2 or 3 times daily to a subject, preferably at least once daily to the subject. In a preferred embodiment of the method, the composition is provided orally.

Particular embodiments of the present disclosure are described in the following numbered paragraphs:

1. A composition comprising one or more fructanases and one or more Lactobacillus strains for a use which is selected from a group consisting of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; and
d. any combination thereof.
   2. The use according to paragraph 1, wherein the Lactobacillus strain is Lactobacillus johnsonii or Lactobacillus crispatus.
   3. The use according to any one of paragraphs 1 or 2, wherein the subject is human.
   4. The use according to any one of the preceding paragraphs, wherein the fructan and/or fructose induced abdominal discomfort is diarrhea, pain, flatulence, nausea, constipation, bloating and/or gas induced from ingestion of products comprising fructan and/or fructose.
   5. The use according to any one of the preceding paragraphs, wherein the composition is provided from 1 to 3 times daily to a subject, preferably at least once daily to a subject.
   6. The use according to any one of the preceding paragraphs, wherein the composition is provided orally to the subject.
   7. The use according to any one of the preceding paragraphs, wherein the composition is provided with or before a meal of the subject.
   8. The use according to any one of the preceding paragraphs, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant of Lactobacillus johnsonii strain deposited under deposit accession number DSM 33901.
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant of Lactobacillus crispatus strain deposited under deposit accession number DSM 34452.
   9. The use according to any one of the preceding paragraphs, wherein the Lactobacillus strains survive in the gastrointestinal tract.
   10. The use according to any one of the preceding paragraphs, wherein the Lactobacillus strains grow on fructose in vitro and/or in the gastrointestinal tract of a subject.
   11. The use according to any one of the preceding paragraphs, wherein the Lactobacillus strains reduce gas production of fecal matter induced with fructan and/or fructose in a gas pressure measurement system.
   12. The use according to any one of the preceding paragraphs, wherein the composition reduces at least 20-50% gas production of fecal matter induced with fructan and/or fructose in a gas pressure measurement system.
   13. The use according to any one of the preceding paragraphs, wherein the composition reduces at least 20% gas production of fecal matter induced with fructan and/or fructose in a gas pressure measurement system.
   14. The use according to any one of the preceding paragraphs, wherein the composition comprises 1E+08 Colony Forming Units (CFU) to 1E+11 CFU of the one or more Lactobacillus strains.
   15. The use according to any one of the preceding paragraphs, wherein the composition comprises about 1E+09 CFU of the one or more Lactobacillus strains.
   16. The use according to any one of the preceding paragraphs, wherein the one or more fructanases belong to the GH32 Family.
   17. The use according to any one of the preceding paragraphs, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).
   18. The use according to any one of the preceding paragraphs, wherein the one or more fructanases is selected from the group consisting of: invertase (EC 3.2.1.26), endo-inulinase (EC 3.2.1.7) and exo-inulinase (EC 3.2.1.80).
   19. The use according to any one of paragraphs 15-16, wherein the one or more fructanases is selected from the group consisting of: endo-inulinase (EC 3.2.1.7) and exo-inulinase (EC 3.2.1.80).
   20. The use according to any one of paragraphs 17-19, wherein the exo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1;
b. a polypeptide derived from SEQ ID NO:1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   21. The use according to paragraph 20, wherein the exo-inulinase is a polypeptide comprising, consisting essentially of, or consisting of SEQ ID NO:1.
   22. The use according to any one of paragraphs 17-19, wherein the endo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2;
b. a polypeptide derived from SEQ ID NO:2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   23. The use according to paragraph 22, wherein the endo-inulinase is a polypeptide comprising, consisting essentially of, or consisting of SEQ ID NO:2.
   24. The use according to any one of paragraphs 17-19, wherein the invertase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:3;
b. a polypeptide derived from SEQ ID NO:3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   25. The use according to paragraph 24, wherein the invertase is a polypeptide comprising, consisting essentially of, or consisting of SEQ ID NO:3.
   26. The use according to any one of the preceding paragraphs, wherein the composition comprises 1 to 5 milligram (mg) enzyme protein, preferably 1.5 to 4.5 mg enzyme protein, preferably 2 to 4 mg enzyme protein, preferably 2.5 to 3.5 mg enzyme protein, preferably 2 to 3 mg enzyme protein of the one or more fructanases.
   27. The use according to paragraph 26, wherein the composition comprises 1.5 to 2.5 mg enzyme protein of the one or more fructanases.
   28. The use according to any one of the preceding paragraphs, wherein the composition comprises 3 to 5 mg enzyme protein, preferably 3.2 to 4.5 mg enzyme protein 1.5 to 2.5 mg enzyme protein of the one or more endo-inulinase.
   29. The use according to any one of the preceding paragraphs, wherein the composition comprises 1.5 to 2 mg enzyme protein, preferably 1.3 to 1.9 mg enzyme protein of the one or more exo-inulinase.
   30. The use according to any one of the preceding paragraphs, wherein the one or more fructanases breaks down fructan to fructose and the one or more Lactobacillus strains breaks down fructose.
   31. The use according to any one of the preceding paragraphs, wherein the one or more fructanases induces the growth of Bifidobacterium during hydrolysis of inulin.
   32. The use according to any one of the preceding paragraphs, wherein the one or more Lactobacillus strains breaks down galactan and/or galactose.
   33. The use according to any one of the preceding paragraphs, wherein the fructan is fructo-oligo saccharides (FOS).
   34. One or more Lactobacillus strains or a composition comprising such for a use which is selected from a group consisting of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; and
d. any combination thereof.
   35. The one or more Lactobacillus strains or a composition comprising such for a use according to paragraph 34, wherein the Lactobacillus strain is Lactobacillus johnsonii or Lactobacillus crispatus.
   36. The one or more Lactobacillus strains or a composition comprising such for a use according to any one of paragraphs 34 or 35, wherein the subject is human.
   37. The one or more Lactobacillus strains or a composition comprising such for a use according to any one of paragraphs 34-36, wherein fructan and/or fructose induced abdominal discomfort is diarrhea, pain, flatulence, nausea, constipation, bloating and/or gas induced from ingestion of products comprising fructan and/or fructose.
   38. The one or more Lactobacillus strains or a composition comprising such for a use according to any one of paragraphs 34-37, wherein the Lactobacillus strains or composition are provided from 1 to 3 times daily to a subject, preferably at least once daily to a subject.
   39. The one or more Lactobacillus strains or a composition comprising such for a use according to any one of paragraphs 34-38, wherein the one or more Lactobacillus strains or the composition is provided orally to the subject.
   40. The one or more Lactobacillus strains or a composition comprising such for a use according to any one of paragraphs 34-39, wherein the composition is provided with or before a meal of the subject.
   41. The one or more Lactobacillus strains or a composition comprising such for a use according to any one of paragraphs 34-40, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant of Lactobacillus johnsonii strain deposited under deposit accession number DSM 33901.
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant of Lactobacillus crispatus strain deposited under deposit accession number DSM 34452.
   42. The one or more Lactobacillus strains or a composition comprising such for a use according to any one of paragraphs 34-41, wherein the Lactobacillus strains survive in the gastrointestinal tract.
   43. The one or more Lactobacillus strains or a composition comprising such for a use according to any one of paragraphs 34-42, wherein the Lactobacillus strains grow on fructose in vitro and/or in the gastrointestinal tract of a subject.
   44. The one or more Lactobacillus strains or a composition comprising such for a use according to any one of paragraphs 34-43, wherein the composition comprises 1E+08 CFU to 1E+11 CFU of the one or more Lactobacillus strains.
   45. The one or more Lactobacillus strains or a composition comprising such for a use according to any one of paragraphs 34-44, wherein the composition comprises about 1E+09 CFU of the one or more Lactobacillus strains.
   46. The one or more Lactobacillus strains or a composition comprising such for a use according to any one of paragraphs 34-45, wherein the composition further comprises one or more fructanases.
   47. The one or more Lactobacillus strains or a composition comprising such for a use according to any one of paragraphs 34-46, wherein the composition reduces gas production of fecal matter induced with fructan and/or fructose in a gas pressure measurement system.
   48. The one or more Lactobacillus strains or a composition comprising such for a use according to any one of paragraphs 34-47, wherein the composition reduces at least 20-50% gas production of fecal matter induced with fructan and/or fructose in a gas pressure measurement system.
   49. The one or more Lactobacillus strains or a composition comprising such for a use according to paragraph 48, wherein the composition reduces at least 20% gas production of fecal matter induced with fructan and/or fructose in a gas pressure measurement system.
   50. One or more fructanases or a composition comprising such for a use which is selected from a group consisting of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; and
d. any combination thereof.
   51. The one or more fructanases or a composition comprising such for a use according to paragraph 50, wherein the subject is human.
   52. The one or more fructanases or a composition comprising such for a use according to paragraphs 50 or 51, wherein the one or more fructanases belong to the GH32 Family.
   53. The one or more fructanases or a composition comprising such for a use according to paragraphs 50-52, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).
   54. The one or more fructanases or a composition comprising such for a use according to paragraph 53, wherein the one or more fructanases is selected from the group consisting of: invertase (EC 3.2.1.26), endo-inulinase (EC 3.2.1.7) and exo-inulinase (EC 3.2.1.80).
   55. The one or more fructanases or a composition comprising such for a use according to any one of paragraphs 50-54, wherein the one or more fructanases is selected from the group consisting of: endo-inulinase (EC 3.2.1.7) and exo-inulinase (EC 3.2.1.80).
   56. The one or more fructanases or a composition comprising such for a use according to any one of paragraphs 50-55, wherein the exo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1;
b. a polypeptide derived from SEQ ID NO:1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   57. The one or more fructanases or a composition comprising such for a use according to paragraph 56, wherein the exo-inulinase is a polypeptide comprising, consisting essentially of, or consisting of SEQ ID NO:1.
   58. The one or more fructanases or a composition comprising such for a use according to any one of paragraphs 50-55, wherein the endo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2;
b. a polypeptide derived from SEQ ID NO:2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   59. The one or more fructanases or a composition comprising such for a use according to paragraph 58, wherein the endo-inulinase is a polypeptide comprising, consisting essentially of, or consisting of SEQ ID NO:2.
   60. The one or more fructanases or a composition comprising such for a use according to any one of paragraphs 50-55, wherein the invertase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:3;
b. a polypeptide derived from SEQ ID NO:3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   61. The one or more fructanases or a composition comprising such for a use according to paragraph 60, wherein the invertase is a polypeptide comprising, consisting essentially of, or consisting of SEQ ID NO:3.
   62. The one or more fructanases or a composition comprising such for a use according to any one of paragraphs 50-62, wherein the composition comprises 1 to 5 mg enzyme protein, preferably 1.5 to 4.5 mg enzyme protein, preferably 2 to 4 mg enzyme protein, preferably 2.5 to 3.5 mg enzyme protein, preferably 2 to 3 mg enzyme protein of the one or more fructanases.
   63. The one or more fructanases or a composition comprising such for a use according to paragraph 62, wherein the composition comprises 1.5 to 2.5 mg enzyme protein of the one or more fructanases.
   64. The one or more fructanases or a composition comprising such for a use according to any one of paragraphs 50-63, wherein the composition comprises 3 to 5 mg enzyme protein, preferably 3.2 to 4.5 mg enzyme protein of the one or more endo-inulinase.
   65. The one or more fructanases or a composition comprising such for a use according to any one of paragraphs 50-55 or 58-59, wherein the composition comprises 1.5 to 2 mg enzyme protein, preferably 1.3 to 1.9 mg enzyme protein of the one or more exo-inulinase.
   66. The one or more fructanases or a composition comprising such for a use according to any one of paragraphs 50-65, wherein the composition further comprises one or more Lactobacillus strains.
   67. The one or more fructanases or a composition comprising such for a use according to any one of paragraphs 50-66, wherein the Lactobacillus strain is Lactobacillus johnsonii or Lactobacillus crispatus.
   68. The one or more fructanases or a composition comprising such for a use according to any one of paragraphs 50-67, wherein the one or more fructanases breaks down fructan to fructose and the one or more Lactobacillus strains breaks down fructose.
   69. The one or more fructanases or a composition comprising such for a use according to any one of paragraphs 50-68, wherein the one or more fructanases induces the growth of Bifidobacterium during hydrolysis of inulin.
   70. The one or more fructanases or a composition comprising such for a use according to any one of paragraphs 50-69, wherein the composition reduces gas production of fecal matter induced with fructan and/or fructose in a gas pressure measurement system.
   71. The one or more fructanases or a composition comprising such for a use according to any one of paragraphs 50-70, wherein the composition reduces at least 20-50% gas production of fecal matter induced with fructan and/or fructose in a gas pressure measurement system.
   72. The one or more fructanases or a composition comprising such for a use according to any one of paragraphs 50-71, wherein the composition reduces at least 20% gas production of fecal matter induced with fructan and/or fructose in a gas pressure measurement system.
   73. A Lactobacillus strain for providing relief from or preventing fructan and/or fructose induced abdominal discomfort, digesting fructan and/or fructose or improving digestion of fructan and/or fructose, and providing digestive comfort after digestion of fructan and/or fructose on consumption of products containing fructan and/or fructose to a subject, wherein the Lactobacillus strain in presence of one or more fructanases results in at least 20-50% reduction of gas pressure development, when tested against the fecal fermentation of the fiber alone across different donors such as across 2-15 different donors, e.g. 2, 3, 4, 5, 6, 7, 8, 9, 10, 11, 12, 13, 14 or 15 different donors.
   74. The Lactobacillus strain according to paragraph 73, wherein the Lactobacillus strain in presence of one or more fructanases results in at least 20% reduction of gas pressure development, when tested against the fecal fermentation of the fiber alone across different donors.
   75. The Lactobacillus strain according to any one of paragraphs 73 or 74, wherein the Lactobacillus strain survives in gastrointestinal tracts.
   76. The Lactobacillus strain according to any one of paragraphs 73-75, wherein the Lactobacillus strain grows on fructose in vitro and/or in the gastrointestinal tract of a subject.
   77. The Lactobacillus strain according to any one of paragraphs 73-76, wherein the Lactobacillus strain in presence of one or more fructanases reduces gas production of fecal matter induced with fructan and/or fructose in a gas pressure measurement system.
   78. The Lactobacillus strain according to any one of paragraphs 73-77, wherein the Lactobacillus strain in presence of one or more fructanases reduces at least 20-50% gas production of fecal matter induced with fructan and/or fructose in a gas pressure measurement system
   79. The Lactobacillus strain according to any one of paragraphs 73-78, wherein the Lactobacillus strain in presence of one or more fructanases reduces at least 20% gas production of fecal matter induced with fructan and/or fructose in a gas pressure measurement system.
   80. The Lactobacillus strain according to any one of paragraphs 73-79, wherein the Lactobacillus strain is Lactobacillus johnsonii or Lactobacillus crispatus
   81. The Lactobacillus strain according to paragraph 80, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant thereof.
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant thereof.
   82. A fructanase for providing relief from or preventing fructan and/or fructose induced abdominal discomfort, digesting fructan and/or fructose or improving digestion of fructan and/or fructose, and providing digestive comfort after digestion of fructan and/or fructose on consumption of products containing fructan and/or fructose to a subject, wherein the fructanase results in at least 20-50% reduction of gas pressure development, when tested against the fecal fermentation of the fiber alone across different donors such as across 2-15 different donors, e.g. 2, 3, 4, 5, 6, 7, 8, 9, 10, 11, 12, 13, 14 or 15 different donors.
   83. The fructanase according to paragraph 82, wherein the subject is human.
   84. The fructanase according to any one of paragraphs 82 or 83, wherein the fructanase results in at least 20% reduction of gas pressure development, when tested against the fecal fermentation of the fiber alone across different donors.
   85. The fructanase according to any one of paragraphs 82-84, wherein the one or more fructanases belong to the GH32 Family.
   86. The fructanase according to any one of paragraphs 82-85, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).
   87. The fructanase according to paragraph 86, wherein the one or more fructanases is selected from the group consisting of: invertase (EC 3.2.1.26), endo-inulinase (EC 3.2.1.7) and exo-inulinase (EC 3.2.1.80).
   88. The fructanase according to any one of paragraphs 79-80, wherein the one or more fructanases is selected from the group consisting of: endo-inulinase (EC 3.2.1.7) and exo-inulinase (EC 3.2.1.80).
   89. The fructanase according to any one of paragraphs 85-88, wherein the exo-inulinase is a polypeptide selected from the group consisting of:
e. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1;
f. a polypeptide derived from SEQ ID NO:1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
g. a polypeptide derived from the polypeptide of (a) or (b), wherein the N- and/or C-terminal end has been extended by addition of one or more amino acids, and
h. a fragment of the polypeptide of (a), (b), or (c).
   90. The fructanase according to paragraph 89, wherein the exo-inulinase is a polypeptide comprising, consisting essentially of, or consisting of SEQ ID NO:1.
   91. The fructanase according to any one of paragraphs 85-88, wherein the endo-inulinase is a polypeptide selected from the group consisting of:
e. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2;
f. a polypeptide derived from SEQ ID NO:2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
g. a polypeptide derived from the polypeptide of (a) or (b), wherein the N- and/or C-terminal end has been extended by addition of one or more amino acids, and
h. a fragment of the polypeptide of (a), (b), or (c).
   92. The fructanase according to paragraph 91, wherein the endo-inulinase is a polypeptide comprising, consisting essentially of, or consisting of SEQ ID NO:2.
   93. The fructanase according to any one of paragraphs 85-88, wherein the invertase is a polypeptide selected from the group consisting of:
e. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:3;
f. a polypeptide derived from SEQ ID NO:3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
g. a polypeptide derived from the polypeptide of (a) or (b), wherein the N- and/or C-terminal end has been extended by addition of one or more amino acids, and
h. a fragment of the polypeptide of (a), (b), or (c).
   94. The fructanase according to paragraph 93, wherein the invertase is a polypeptide comprising, consisting essentially of, or consisting of SEQ ID NO:3.
   95. A composition for providing relief from or preventing fructan and/or fructose induced abdominal discomfort, digesting fructan and/or fructose or improving digestion of fructan and/or fructose, and providing digestive comfort after digestion of fructan and/or fructose on consumption of products containing fructan and/or fructose to a subject, the composition comprising:
(a) one or more Lactobacillus strains; and
(b) one or more fructanases.
   96. The composition according to paragraph 95, wherein the Lactobacillus strain is Lactobacillus johnsonii or Lactobacillus crispatus.
   97. The composition according to any one of paragraphs 95 or 96, wherein the subject is human.
   98. The composition according to paragraph 95, wherein fructan and/or fructose induced abdominal discomfort is diarrhea, pain, flatulence, nausea, constipation, bloating and/or gas induced from ingestion of products comprising fructan and/or fructose.
   99. The composition according to any one of paragraphs 95-98, wherein the Lactobacillus strains or composition are provided from 1 to 3 times daily to a subject, preferably at least once daily to a subject.
   100. The composition according to any one of paragraphs 95-99, wherein the one or more Lactobacillus strains or the composition is provided orally to the subject.
   101. The composition according to any one of paragraphs 95-100, wherein the composition is provided with or before a meal of the subject.
   102. The composition according to paragraphs 95-101, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant of Lactobacillus johnsonii strain deposited under deposit accession number DSM 33901.
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant of Lactobacillus crispatus strain deposited under deposit accession number DSM 34452.
   103. The composition according to paragraph 102, wherein the Lactobacillus strains survive in the gastrointestinal tract.
   104. The composition according to any one of paragraphs 95-103, wherein the Lactobacillus strains grow on fructose in vitro and/or in the gastrointestinal tract of a subject.
   105. The composition according to any one of paragraphs 95-104, wherein the Lactobacillus strains reduce gas production of fecal matter induced with fructan and/or fructose in a gas pressure measurement system.
   106. The composition according to any one of paragraphs 95-105, wherein the composition reduces at least 20-50% gas production of fecal matter induced with fructan and/or fructose in a gas pressure measurement system
   107. The composition according to any one of paragraphs 95-106, wherein the composition reduces at least 20% gas production of fecal matter induced with fructan and/or fructose in a gas pressure measurement system.
   108. The composition according to any one of paragraphs 95-107, wherein the composition is in a single dosage form or, a multiple dosage form.
   109. The composition according to paragraph 108, wherein the multiple dosage form is one dosage form for the one or more fructanases and another dosage form for the Lactobacillus strains.
   110. The composition according to paragraph 109, wherein the dosage form of the composition is capsules, powders, microcapsules, tablets, dragees, pellets granules, chewables and liquid forms.
   111. The composition according to any one of paragraphs 95-110, wherein the composition comprises 1E+08 CFU to 1E+11 CFU of the one or more Lactobacillus strains.
   112. The composition according to paragraph 111, wherein the composition comprises about 1E+09 CFU of the one or more Lactobacillus strains.
   113. The composition according to any one of paragraphs 95-112, wherein the one or more fructanases belong to the GH32 Family.
   114. The composition according to any one of paragraphs 95-113, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).
   115. The composition according to paragraph 114, wherein the one or more fructanases is selected from the group consisting of: invertase (EC 3.2.1.26), endo-inulinase (EC 3.2.1.7) and exo-inulinase (EC 3.2.1.80).
   116. The composition according to any one of paragraphs 114-115, wherein the one or more fructanases is selected from the group consisting of: endo-inulinase (EC 3.2.1.7) and exo-inulinase (EC 3.2.1.80).
   117. The composition according to any one of paragraphs 95-116, wherein the composition comprises 1 to 5 mg enzyme protein, preferably 1.5 to 4.5 mg enzyme protein, preferably 2 to 4 mg enzyme protein, preferably 2.5 to 3.5 mg enzyme protein, preferably 2 to 3 mg enzyme protein of the one or more fructanases.
   118. The composition according to any one of paragraphs 95-117, wherein the composition comprises 1.5 to 2.5 mg enzyme protein of the one or more fructanases.
   119. The composition according to any one of paragraphs 114-118, wherein the exo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1,
b. a polypeptide derived from SEQ ID NO:1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   120. The composition according to paragraph 119, wherein the exo-inulinase is a polypeptide comprising, consisting essentially of, or consisting of SEQ ID NO:1.
   121. The composition according to any one of paragraphs 114-116 or 119-120, wherein the composition comprises 1.5 to 2 mg enzyme protein, preferably 1.3 to 1.9 mg enzyme protein of the one or more exo-inulinase.
   122. The composition according to any one of paragraphs 114-116, wherein the endo-inulinase is a polypeptide selected from the group consisting of:
e. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2;
f. a polypeptide derived from SEQ ID NO:2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
g. a polypeptide derived from the polypeptide of (a) or (b), wherein the N- and/or C-terminal end has been extended by addition of one or more amino acids, and
h. a fragment of the polypeptide of (a), (b), or (c).
   123. The composition according to paragraph 122, wherein the endo-inulinase is a polypeptide comprising, consisting essentially of, or consisting of SEQ ID NO:2.
   124. The composition according to any one of paragraphs 114-116 or 122-123, wherein the composition comprises 3 to 5 mg enzyme protein, preferably 3.2 to 4.5 mg enzyme protein of the one or more endo-inulinase.
   125. The composition according to any one of paragraphs 114-116, wherein the invertase is a polypeptide selected from the group consisting of:
i. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:3;
j. a polypeptide derived from SEQ ID NO:3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
k. a polypeptide derived from the polypeptide of (a) or (b), wherein the N- and/or C-terminal end has been extended by addition of one or more amino acids, and
l. a fragment of the polypeptide of (a), (b), or (c).
   126. The composition according to paragraph 125, wherein the invertase is a polypeptide comprising, consisting essentially of, or consisting of SEQ ID NO:3.
   127. The composition according to any one of paragraphs 95-126, wherein the one or more fructanases breaks down fructan to fructose and the one or more Lactobacillus strains breaks down fructose.
   128. The composition according to any one of paragraphs 95-127, wherein the one or more fructanases induces the growth of Bifidobacterium during hydrolysis of inulin.
   129. A method for providing relief from or preventing fructan and/or fructose induced abdominal discomfort, digesting fructan and/or fructose or improving digestion of fructan and/or fructose, and providing digestive comfort after digestion of fructan and/or fructose on consumption of products containing fructan and/or fructose to a subject; the method comprising providing to a subject in need thereof an effective amount a composition comprising one or more fructanases and one or more Lactobacillus strains.
   130. The method according to paragraph 129, wherein the Lactobacillus strain is Lactobacillus johnsonii or Lactobacillus crispatus.
   131. The method according to any one of paragraphs 129 or 130, wherein the subject is human.
   132. The method according to any one of paragraphs 129-131, wherein the fructan and/or fructose induced abdominal discomfort is diarrhea, pain, flatulence, nausea, constipation, bloating and/or gas induced from ingestion of products comprising fructan and/or fructose.
   133. The method according to any one of paragraphs 129-132, wherein the Lactobacillus strains or composition are provided from 1 to 3 times daily to a subject, preferably at least once daily to a subject.
   134. The method according to any one of paragraphs 129-133, wherein the composition is provided orally to the subject.
   135. The method according to any one of paragraphs 129-134, wherein the composition is provided with or before a meal of the subject.
   136. The method according to any one of paragraphs 129-135, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant of Lactobacillus johnsonii strain deposited under deposit accession number DSM 33901.
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant of Lactobacillus crispatus strain deposited under deposit accession number DSM 34452.
   137. The method according to any one of paragraphs 129-136, wherein the Lactobacillus strains survive in the gastrointestinal tract.
   138. The method according to any one of paragraphs 129-137, wherein the Lactobacillus strains grow fructose in vitro and/or in the gastrointestinal tract of a subject.
   139. The method according to any one of paragraphs 129-138, wherein the Lactobacillus strains reduce gas production of fecal matter induced with fructan and/or fructose in a gas pressure measurement system.
   140. The method according to any one of paragraphs 129-139, wherein the composition reduces at least 20-50% gas production of fecal matter induced with fructan and/or fructose in a gas pressure measurement system.
   141. The method according to any one of paragraphs 129-140, wherein the composition reduces at least 20% gas production of fecal matter induced with fructan and/or fructose in a gas pressure measurement system.
   142. The method according to any one of paragraphs 129-141, wherein the composition comprises 1E+08 CFU to 1E+11 CFU of the one or more Lactobacillus strains.
   143. The method according to paragraph 142, wherein the composition comprises about 1E+09 CFU of the one or more Lactobacillus strains.
   144. The method according to any one of paragraphs 129-143, wherein the one or more fructanases belong to the GH32 Family.
   145. The method according to any one of paragraphs 129-144, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).
   146. The method according to paragraph 145, wherein the one or more fructanases is selected from the group consisting of: invertase (EC 3.2.1.26), endo-inulinase (EC 3.2.1.7) and exo-inulinase (EC 3.2.1.80).
   147. The method according to any one of paragraphs 144-146, wherein the one or more fructanases is selected from the group consisting of: endo-inulinase (EC 3.2.1.7) and exo-inulinase (EC 3.2.1.80).
   148. The method according to any one of paragraphs 144-147, wherein the exo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1,
b. a polypeptide derived from SEQ ID NO:1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   149. The method according to paragraph 148, wherein the exo-inulinase is a polypeptide comprising, consisting essentially of, or consisting of SEQ ID NO:1.
   150. The method according to any one of paragraphs 144-147, wherein the endo-inulinase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2;
b. a polypeptide derived from SEQ ID NO:2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   151. The method according to paragraph 150, wherein the endo-inulinase is a polypeptide comprising, consisting essentially of, or consisting of SEQ ID NO:2.
   152. The method according to any one of paragraphs 144-147, wherein the invertase is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:3;
b. a polypeptide derived from SEQ ID NO:3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).
   153. The method according to paragraph 152, wherein the invertase is a polypeptide comprising, consisting essentially of, or consisting of SEQ ID NO:3.
   154. The method according to any one of paragraphs 129-153, wherein the composition comprises 1 to 5 mg enzyme protein, preferably 1.5 to 4.5 mg enzyme protein, preferably 2 to 4 mg enzyme protein, preferably 2.5 to 3.5 mg enzyme protein, preferably 2 to 3 mg enzyme protein of the one or more fructanases.
   155. The method according to paragraph 154, wherein the composition comprises 1.5 to 2.5 mg enzyme protein of the one or more fructanases.
   156. The method according to any one of paragraphs 144-149, wherein the composition comprises 1.5 to 2 mg enzyme protein, preferably 1.3 to 1.9 mg enzyme protein of the one or more exo-inulinase.
   157. The method according to any one of paragraphs 144-147 or 150-151, wherein the composition comprises 3 to 5 mg enzyme protein, preferably 3.2 to 4.5 mg enzyme protein of the one or more endo-inulinase.
   158. The method according to any one of paragraphs 129-157, wherein the composition is provided 1 to 3 times daily to a subject, preferably daily at least once to the subject.
   159. The method according to any one of paragraphs 129-158, wherein the composition is provided orally to the subject.
   160. The method according to any one of paragraphs 129-159, wherein the composition is provided orally to the subject.
   161. The method according to any one of paragraphs 129-160, wherein the one or more fructanases breaks down fructan to fructose and the one or more Lactobacillus strains breaks down fructose.
   162. The method according to any one of paragraphs 129-161, wherein the one or more fructanases induces the growth of Bifidobacterium during hydrolysis of inulin.

### EXAMPLES

The following examples are not intended to be a detailed catalogue of all the different ways in which the present disclosure may be implemented or of all the features that may be added to the present disclosure. Subjects skilled in the art will appreciate that numerous variations and additions to the various embodiments may be made without departing from the present disclosure. Hence, the following descriptions are intended to illustrate some particular embodiments of the invention and not to exhaustively specify all permutations, combinations and variations thereof.

Unless otherwise indicated, the percentages set forth in the following examples are by weight, based upon the total weight of the composition.

### Material and Methods

Standard media were used for growing Lactobacillus strains, for example a medium providing 15-30 g peptone or tryptone per litre and 5-30 g glucose per litre. For preparing the standard media and the standard procedure was applied (concentration of components, sterilization, inoculation) and the microorganism was allowed to grow at elevated temperature (e.g. 35-42° C.) for sufficient time (e.g. 8-48 hours) unless otherwise stated.

### Example 1—Fructanases

The enzymes tested herein are:
Enzyme A: Multicomponent product Fructozyme LTM comprising Enzyme B, Enzyme C and Enzyme D from Aspergillus Niger, obtained from Novozymes A/S, Denmark.
Enzyme B: Exo-inulinase having the mature amino acid sequence of SEQ ID NO:1
Enzyme C: Endo-inulinase having the mature amino acid sequence of SEQ ID NO:2
Enzyme D: Invertase having the mature amino acid sequence of SEQ ID NO:3
Enzyme E: Bacterial fructanase product BacTop
Enzyme F: Inulinase P obtained from Bio-Cat, Virginia, USA

### Example 2—Screening 1 for Suitable Lactobacillus Strains

Lactobacillus strains having a beneficial impact on the gut, such as reducing gut discomfort, were found using the screening protocol outlined below.

### 1. Prescreening

960 Lactobacillus strains were screened by analyzing the optical density at 600 nm (OD600) as an indicator on the pressure reduction potential of the strains in an endpoint growth assay on fructose. For the growth assay, Lactobacillus strains were preincubated as pre- and main culture under standard conditions for 24 hours. Afterwards, Lactobacillus strains were incubated in 1:4 diluted media in absence of any carbohydrate or in presence of fructose or glucose (5 g/L) for 24 hours measuring the OD600 before and after incubation.
13 Lactobacillus strains with OD600 similar for fructose as for glucose were selected from the prescreening.

### 2. Screening

The 13 Lactobacillus strains selected in the prescreening were subjected to a kinetic growth assay that was identical to the prescreening except that selected strains grew in absence of any carbohydrate or in presence of fructose, glucose, fructo-oligo saccharide (FOS) and inulin (5 g/L). The OD600 was continuously measured every 5 min over 24 hours.

One Lactobacillus strain with growth similar for fructose as for glucose as well as with potential to grow on FOS and/or inulin was selected from the screening.

### 3. Hit Confirmation

The Lactobacillus strain selected in the screening was subjected to a kinetic growth assay that was identical to the screening except that no main culture was set up, selected strains grew in 1:2 diluted medium containing either 10 g/L carbohydrate or no carbohydrate and the OD600 was measured as endpoint at the end of the 20 h incubation. Two panels of carbohydrates were tested. Panel 1 contained glucose, fructose, fructo-oligo saccharide (FOS) and inulin, while panel 1 contained glucose, fructose, FOS, saccharose, raffinose, stachyose and galactose.

### Results:

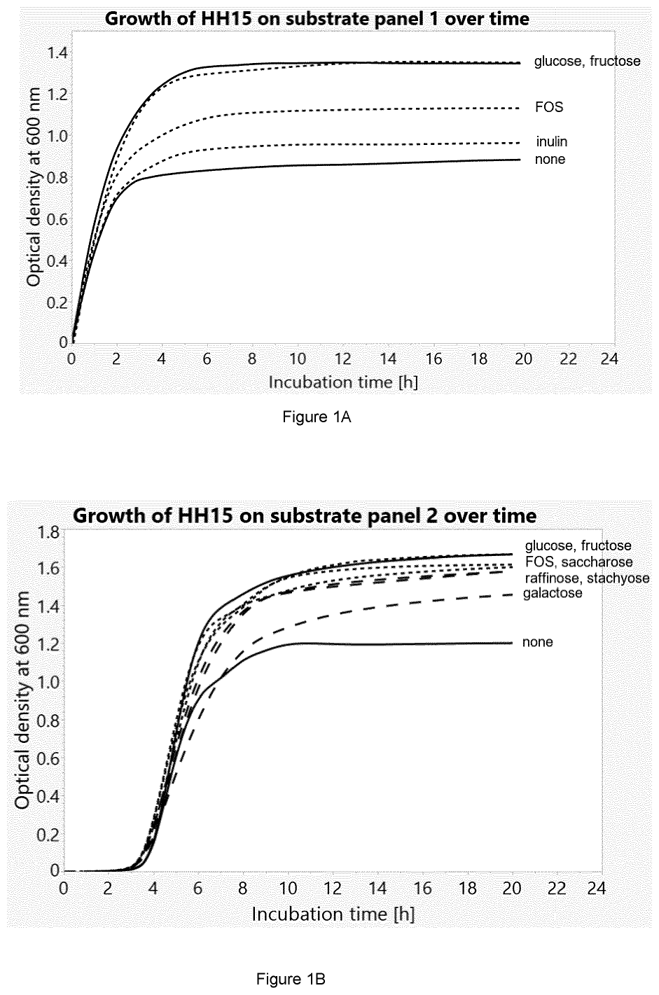

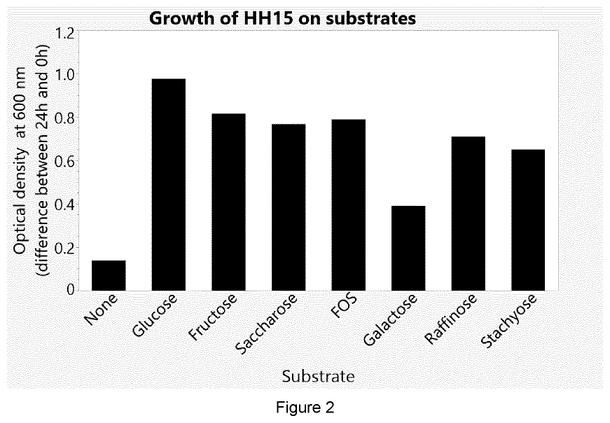

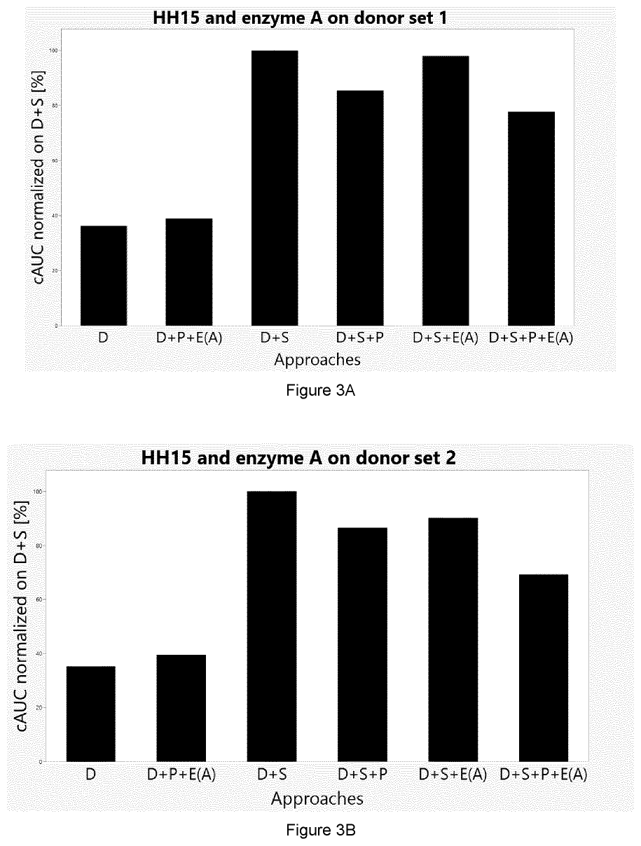

The kinetic and endpoint growth data shows that the selected Lactobacillus strain grows on fructose as well as on glucose (FIGS. 1 and 2). Furthermore, the selected Lactobacillus strain shows the potential to grow on saccharose, FOS, raffinose, stachyose, galactose and inulin compared to the background growth of HH15.

### Example 3—Gas Pressure Tests of Bacterial Fructanases and Invertases

The enzymes enzyme A, enzyme B, enzyme C, Enzyme D, enzyme E, and enzyme F listed in Example 1 were tested in a pressure measurement assay with fecal samples. The pressure was continuously measured within the observation period using inhouse developed pressure measuring devices (2ML-FPM).

Human fecal samples were collected using Gut Alive Kits. The human fecal samples were processed within two days after collecting by diluting fecal samples with glycerol/water in the ratio of 1:4 resulting in 25% fecal samples stored at −80° C. after preparation until using. Inulin (from Chicory) is Substrate (S). The human fecal samples were resuspended in phosphate-buffered saline (PBS) and 5 grams per litre (g/l) Substrate (S) was added. The diluted human fecal samples are termed as Donors (D). There were different donor sets:
a. Donor set 1 had fifteen different fecal donors
b. Donor set 2 had fourteen different fecal donors where one outlier was excluded from donor set 1
c. Donor set 3 had three different fecal donors
d. Donor set 4 had two different fecal donors
e. Donor set 5 had seven different fecal donors
f. Donor set 6 had one fecal donor
g. Donor set 7 had two different fecal donors
h. Donor set 8 had 5 different fecal donors (subset of 15 fecal donors in donor set 1)

The Lactobacillus strain HH15 screened in Example 2 is termed as Probiotic (P). Enzymes listed in Example 1 are termed as Enzyme (E) with concentration as listed in Table 2. Different mixes were prepared with of human fecal samples such as Donor (D), Donor+Probiotic+Enzyme (D+P+E), Donor+Substrate (D+S), and Donor+Substrate+Enzyme (D+S+E) which are called approaches. The approaches Donor (D), Donor+Probiotic+Enzyme (D+P+E), Donor+Substrate (D+S), and Donor+Substrate+Enzyme (D+S+E) shown in table 1 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. For normalization, the cumulative area under the curve (cAUC) was calculated at every measure cycle. The cAUC of approach D+S was set to 100% and the cAUC of all other approaches was set in relation to this.

TABLE 1

| | Fecal | | Strain | Enzyme | |
|---|---|---|---|---|---|
| | sample | Substrate | 1E+8 | Concentration | |
| | 2.5% | 5 g/L | CFU/mL | as listed in | |
| Approach | final | final | final | Table 2 | PBS |
| D | Yes | No | No | No | Yes |
| D + P + E | Yes | No | Yes | Yes | Yes |
| D + S | Yes | Yes | No | No | Yes |
| D + S + P | Yes | Yes | Yes | No | Yes |

TABLE 2

| | | Enzyme |
|---|---|---|
| Enzyme | | Concentration |
| name | Approaches | final |
| E(A) | D + P + E, D + S + E | 1.5E−2 g/L |
| E(B) | D + P + E, D + S + E | 1.5E−3 g/L |
| E(C) | D + P + E, D + S + E | 1.5E−3 g/L |
| E(D) | D + P + E, D + S + E | 7.5E−4 g/L |
| E(E) | D + P + E, D + S + E | 1.5E−2 g/L |
| E(F) | D + P + E, D + S + E | 1.5E−2 g/L |

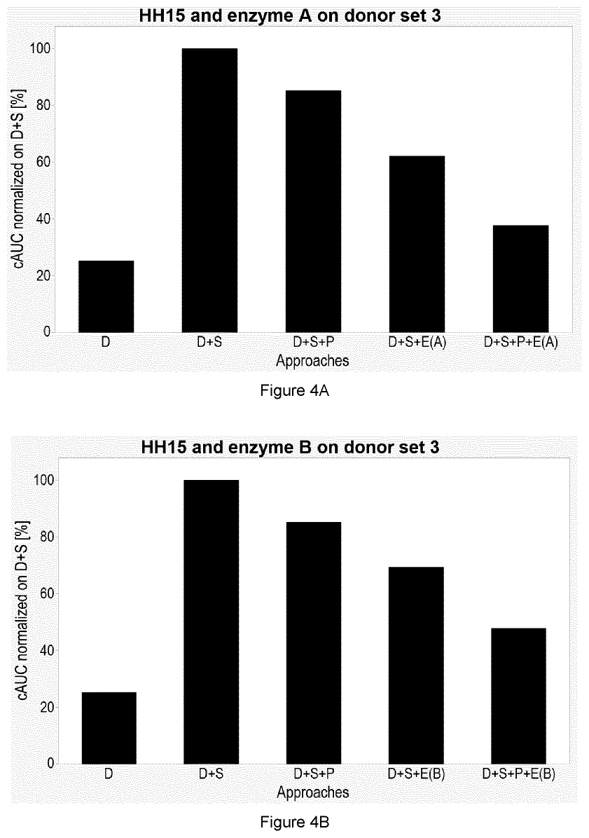

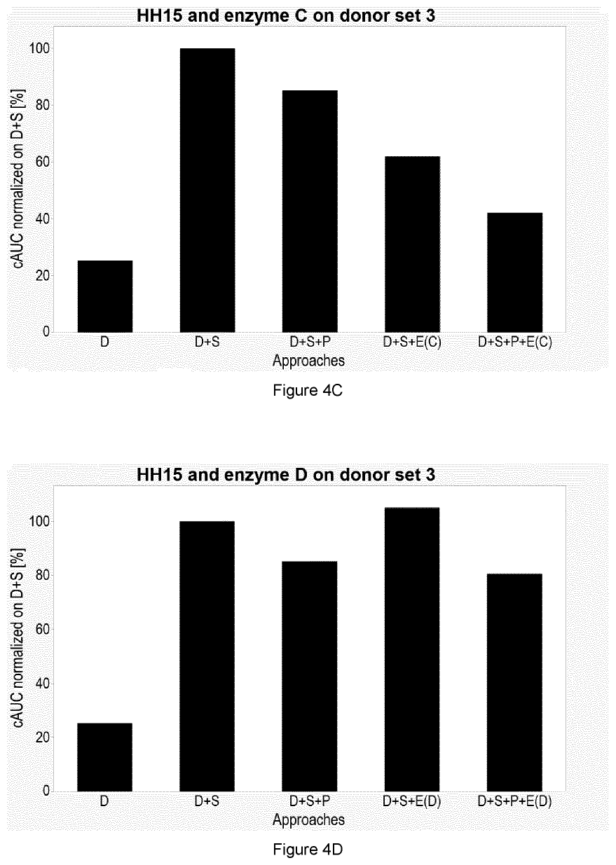

For FIG. 3A, the pressure was calculated as average and represented in a bar chart as cumulated area under the curve (cAUC) in presence of human fecal donor set 1 after 22 h of incubation. Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). Each column represents the mean of fifteen fecal donors each tested one to two times.

Results shown in FIG. 3B is different from FIG. 3A as one outlier was excluded from donor set 1 and the results are for corrected donor set 2 that consists of fourteen different decal donors. An outlier Box Plot test was performed as performed by a person skilled in the art. The outlier Box Plot test identified one of the tested donors, within the D+S+P+E approach, as an outlier. Based on the finding, the corresponding donor was excluded from additional data evaluation. Each column in FIG. 3B represents the mean of fourteen fecal donors each tested one to two times. No additional changes were taken.

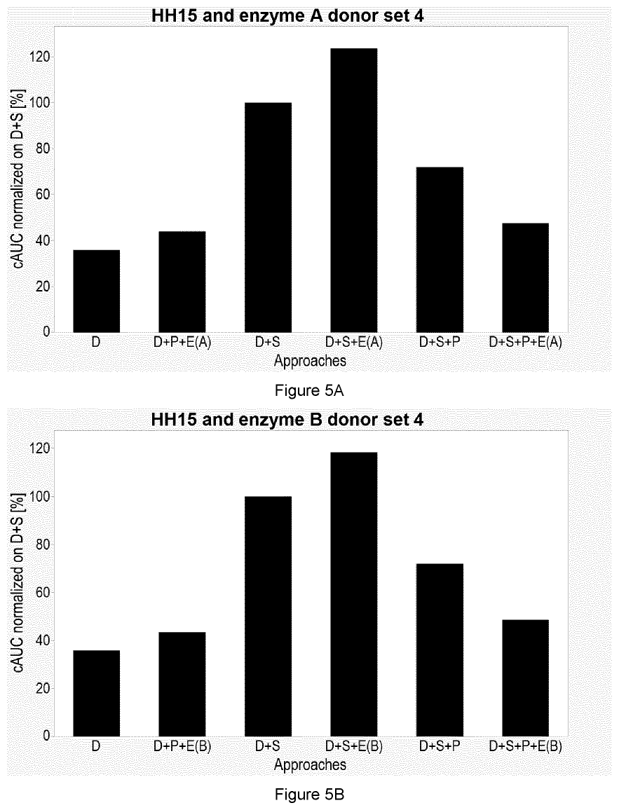

In FIG. 4, the pressure was calculated as average and represented in a bar chart as cumulated area under the curve (cAUC) in presence of human fecal donor set 3 after 22 h of incubation. A) Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). B) Identical with A); however, Enzyme E(A) was substituted by Enzyme E(B). C) Identical with A); however, Enzyme E(A) was substituted by Enzyme E(C).

D) Identical with A); however, Enzyme E(A) was substituted by Invertase E(D). Each column represents the mean of three fecal donors each tested two to four times.

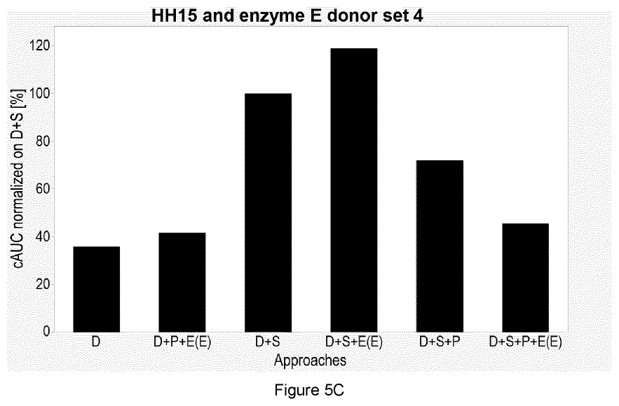

In FIG. 5, the pressure was calculated and represented in a bar chart as cumulated area under the curve (cAUC) in presence of human fecal donor set 4 after 22 h of incubation. A) Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). B) Identical with A); however, enzyme E(A) was substituted by enzyme E(B). C) Identical with A); however, enzyme E(A) was substituted by enzyme E(E). Each column represents the mean of two fecal donors each tested one time.

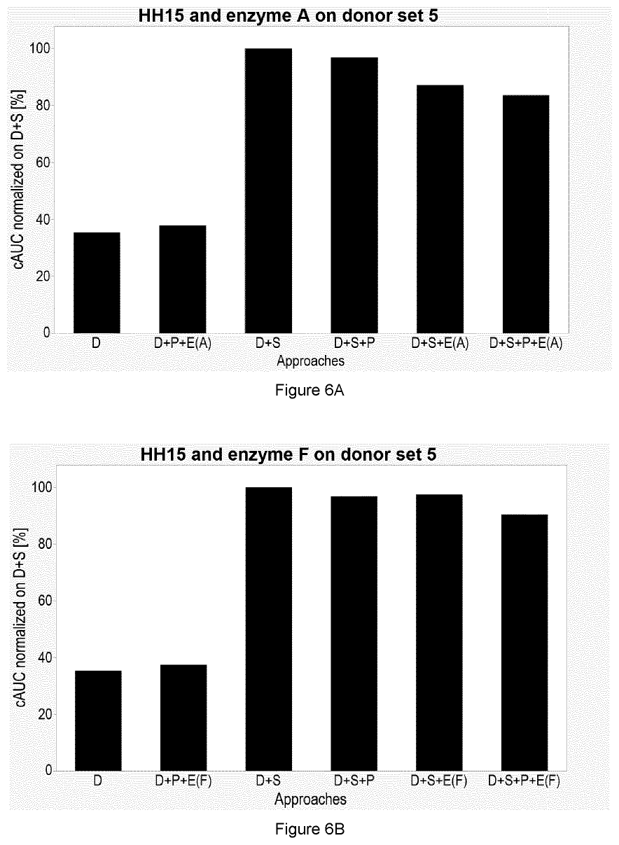

In FIG. 6, the pressure was calculated and represented in a bar chart as cumulated area under the curve (cAUC) in presence of human fecal donor set 5 after 22 h of incubation. A) Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). B) Identical with A); however, enzyme E(A) was substituted by enzyme E(F). Each column represents the mean of seven fecal donors each tested one to two times.

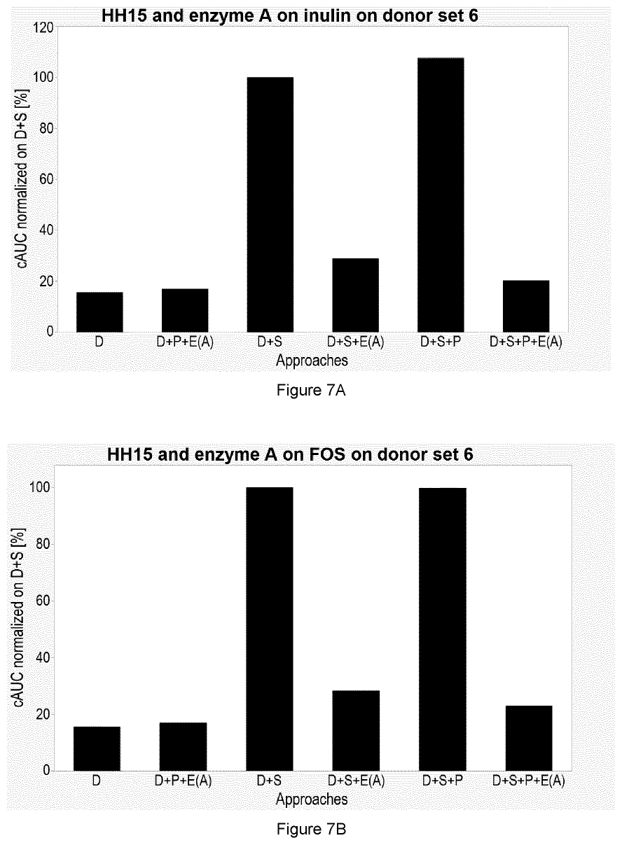

In FIG. 7, the pressure was calculated and represented in a bar chart as cumulated area under the curve (cAUC) in presence of human fecal donor set 6 after 22 h of incubation. A) Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)) with inulin as substrate S. B) Identical with A); however, the substrate inulin (S) was substituted by the substrate FOS (S). Each column represents the mean of duplicates.

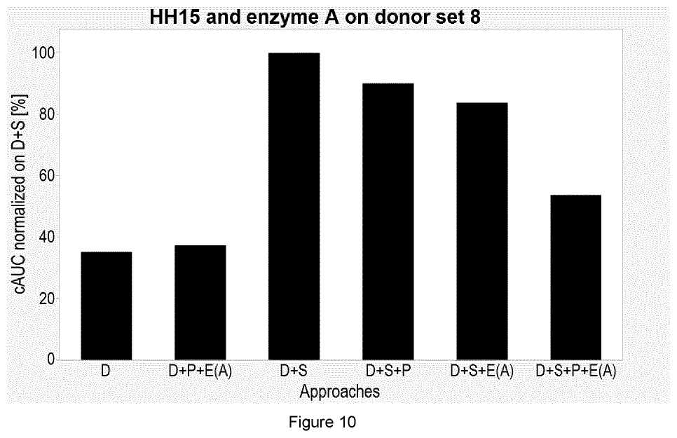

In FIG. 10, the pressure was calculated as average and represented in a bar chart as cumulated area under the curve (cAUC) in presence of human fecal donor set 8 after 22 h of incubation. Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). Each column represents the mean of five fecal donors each tested ones.

Results shown in FIG. 3, FIG. 4, FIG. 5, FIG. 6, FIG. 7 and FIG. 10 show that the chosen donor sets had a substantial influence on the possible degree of pressure reduction. Therefore, it is important to use the same donor for each set of comparison.

Results shown in FIG. 3, FIG. 4, FIG. 5, FIG. 6, FIG. 7, and FIG. 10 show that enzyme A, enzyme B, enzyme C, enzyme E, and enzyme F in approach D+S+E were able to reduce the pressure calculated as cAUC compared to the pressure calculated as cAUC compared to the pressure calculated as cAUC of the D+S approach on different fecal donor sets on average.

Results shown in FIG. 3 show that enzyme A in approach D+S+E was able to reduce the pressure calculated as cAUC compared to the pressure calculated as cAUC of the D+S approach on fecal donor set 1 and donor set 2 (FIGS. 3A and 3B) comparably to one another.

Results shown in FIG. 4 show that enzyme A, enzyme B, and enzyme C in approach D+S+E were able to reduce the pressure calculated as cAUC compared to the pressure calculated as cAUC of the D+S approach on fecal donor set 3, while enzyme D was not able to reduce the pressure calculated as cAUC compared to the pressure calculated as cAUC for the D+S approach on the same fecal donor set.

Results shown in FIG. 5 show that enzyme A, enzyme B, and enzyme E in approach D+S+E changed the pressure calculated as cAUC compared to the pressure calculated as cAUC of the D+S approach on fecal donor set 4 comparably to one another.

Results shown in FIG. 6 show that enzyme A, and enzyme F in approach D+S+E were able to reduce the pressure calculated as cAUC compared to the pressure calculated as cAUC of the D+S approach on donor set 5.

Results shown in FIG. 7 show that enzyme A was able to reduce the pressure calculated as cAUC compared to the pressure calculated as cAUC of the D+S approach on donor set 6.

### Example 4—Gas Pressure Tests of Lactobacillus Strain

The strain HH15 selected in Example 2 was further tested in a pressure measurement assay with fecal samples. The pressure was continuously measured within the observation period using in-house developed pressure measuring devices (2ML-FPM).

Human fecal samples were collected, treated and stored as described in Example 3. The approaches Donor (D), Donor+Probiotic+Enzyme (D+P+E), Donor+Substrate (D+S), and Donor+Substrate+Probiotic (D+S+P) shown in table 3 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. For normalization, the cumulative area under the curve (cAUC) was calculated at every measure cycle. The cAUC of approach D+S was set to 100% and the cAUC of all other approaches was set in relation to this.

TABLE 3

| | Fecal | | Strain | Enzyme | |
|---|---|---|---|---|---|
| | sample | Substrate | 1E+8 | Concentration | |
| | 2.5% | 5 g/L | CFU/mL | as listed in | |
| Approach | final | final | final | Table 4 | PBS |
| D | Yes | No | No | No | Yes |
| D + P + E | Yes | No | Yes | Yes | Yes |
| D + S | Yes | Yes | No | No | Yes |
| D + S + P | Yes | Yes | Yes | No | Yes |

TABLE 4

| | | Enzyme |
|---|---|---|
| Enzyme | | Concentration |
| name | Approachs | final |
| E(A) | D + P + E | 1.5E−2 g/L |
| E(B) | D + P + E | 1.5E−3 g/L |
| E(C) | D + P + E | 1.5E−3 g/L |
| E(D) | D + P + E | 7.5E−4 g/L |
| E(E) | D + P + E | 1.5E−2 g/L |
| E(F) | D + P + E | 1.5E−2 g/L |

Results shown in FIG. 3, FIG. 4, FIG. 5, FIG. 6, FIG. 7 and FIG. 10 show that the chosen donor sets had a substantial influence on the possible degree of pressure reduction. Therefore, it is important to use the same donor for each set of comparison.

Results shown in FIG. 3, FIG. 4, FIG. 5, FIG. 6, and FIG. 7 show that HH15 in the approach D+S+P was able to reduce the pressure calculated as cAUC compared to the pressure calculated as cAUC of the D+S approach on different fecal donor sets on average.

### Example 5—Gas Pressure Tests of Bacterial Fructanases and Lactobacillus Strain in Combination

The strain HH15 tested in Example 4 and the enzymes tested in Example 3 were further tested in combination with each other in a pressure measurement assay with fecal samples. The pressure was continuously measured within the observation period using inhouse developed pressure measuring devices (2ML-FPM).

Human fecal samples were collected, treated and stored as described in Example 3. The approaches Donor (D), Donor+Probiotic+Enzyme (D+P+E), Donor+Substrate (D+S), and Donor+Substrate+Probiotic+Enzyme (D+S+P+E) shown in table 5 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. For normalization, the cumulative area under the curve (cAUC) was calculated at every measure cycle. The cAUC of approach D+S was set to 100% and the cAUC of all other approaches was set in relation to this.

TABLE 5

| | Fecal | | Strain | Enzyme | |
|---|---|---|---|---|---|
| | sample | Substrate | 1E+8 | Concentration | |
| | 2.5% | 5 g/L | CFU/mL | listed in | |
| Approach | final | final | final | Table 6 | PBS |
| D | Yes | No | No | No | Yes |
| D + P + E | Yes | No | Yes | Yes | Yes |
| D + S | Yes | Yes | No | No | Yes |
| D + S + P + E | Yes | Yes | Yes | Yes | Yes |

TABLE 6

| | | Enzyme |
|---|---|---|
| Enzyme | | Concentration |
| name | Approachs | final |
| E(A) | D + P + E, D + S + P + E | 1.5E−2 g/L |
| E(B) | D + P + E, D + S + P + E | 1.5E−3 g/L |
| E(C) | D + P + E, D + S + P + E | 1.5E−3 g/L |
| E(D) | D + P + E, D + S + P + E | 7.5E−4 g/L |
| E(E) | D + P + E, D + S + P + E | 1.5E−2 g/L |
| E(F | D + P + E, D + S + P + E | 1.5E−2 g/L |

Results shown in FIG. 3, FIG. 4, FIG. 5, FIG. 6, FIG. 7 and FIG. 10 show that the chosen donor sets had a substantial influence on the possible degree of pressure reduction. Therefore, it is important to use the same donor for each set of comparison.

Results shown in FIG. 3, FIG. 4, FIG. 5, and FIG. 6 and FIG. 7 show that the combinations of the enzymes A, B, C, E or F with the strain HH15 in approach D+S+P+E were able to reduce the pressure calculated as cAUC compared to the pressure calculated as cAUC in the corresponding D+S approaches on average.

Results shown in FIG. 3, FIG. 4, FIG. 5, FIG. 6, FIG. 7 show that the combinations of the enzymes A, B, C, E or F with the strain HH15 in approach D+S+P+E were able to reduce the pressure calculated as cAUC the most, compared to the pressure calculated as cAUC in the corresponding D+S+P and D+S+E approaches on average.

### Example 6—Gas Pressure Tests on Different Fructans

The strain HH15 tested in Example 4 and the enzyme A tested in Example 3 were further tested as the strain alone, the enzyme alone and in combination with each other in presence of the fructan inulin or fructan FOS (substrate) in a pressure measurement assay with fecal samples. The pressure was continuously measured within the observation period using inhouse developed pressure measuring devices (2ML-FPM).

Human fecal samples were collected, treated and stored as described in Example 3. The approaches Donor (D), Donor+Probiotic+Enzyme (D+P+E), Donor+Substrate (D+S), Donor+Substrate+Probiotic (D+S+P), Donor+Substrate+Enzyme (D+S+E), and Donor+Substrate+Probiotic+Enzyme (D+S+P+E) shown in table 7 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. Either the fructans inulin or FOS were used as substrate S. For normalization, the cumulative area under the curve (cAUC) was calculated at every measure cycle. The cAUC of approach D+S was set to 100% and the cAUC of all other approaches was set in relation to this.

TABLE 7

| | Fecal | | Strain | Enzyme | |
|---|---|---|---|---|---|
| | sample | Substrate | 1E+8 | 1.5 | |
| | 2.5% | 5 g/L | CFU/mL | E−2 g/L | |
| Approach | final | final | final | final | PBS |
| D | Yes | No | No | No | Yes |
| D + P + E | Yes | No | Yes | Yes | Yes |
| D + S | Yes | Yes | No | No | Yes |
| D + S + P | Yes | Yes | Yes | No | Yes |
| D + S + E | Yes | Yes | No | Yes | Yes |
| D + S + P + E | Yes | Yes | Yes | Yes | Yes |

Results shown in FIG. 3, FIG. 4, FIG. 5, FIG. 6, FIG. 7, and FIG. 10 show that the chosen donor sets had a substantial influence on the possible degree of pressure reduction. Therefore, it is important to use the same donor for each set of comparison.

Results shown in FIG. 7 show that the pressure reduction activity of both the strain HH15 alone, the enzyme A alone, and in combination with each other is independent of the chain length of the fructans like inulin and FOS.

### Example 71—Screening 2 for Suitable Lactobacillus Strains

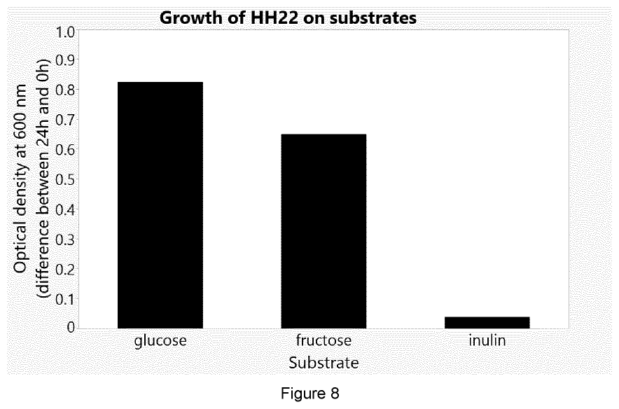

Lactobacillus strains having a beneficial impact on the gut, such as reducing gut discomfort, were found using the screening protocol outlined in Example 2. 1 Lactobacillus strain, hereinafter referred to as HH22, was selected from the set of 960 Lactobacillus strains. Lactobacillus strain HH22, with an OD600 similar for fructose as for glucose as well as potential growth on inulin was selected from the screening (FIG. 8).

### Example 8—Gas Pressure Tests of Lactobacillus Strain

The strain HH22 selected in Example 7 was further tested in a pressure measurement assay with fecal samples. The pressure was continuously measured within the observation period using inhouse developed pressure measuring devices (2ML-FPM).
Human fecal samples were collected, treated and stored as described in Example 3. The Lactobacillus strain HH22 screened in Example 7 is termed as Probiotic (P) in this.
The approaches Donor (D), Donor+Probiotic+Enzyme (D+P+E), Donor+Substrate (D+S), and Donor+Substrate+Probiotic (D+S+P) shown in table 8 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. For normalization, the cumulative area under the curve (cAUC) was calculated at every measure cycle. The cAUC of approach D+S was set to 100% and the cAUC of all other approaches was set in relation to this.

TABLE 8

| | Fecal | | Strain | Enzyme | |
|---|---|---|---|---|---|
| | sample | Substrate | 1E+8 | 1.5E−2 | |
| | 2.5% | 5 g/L | CFU/mL | g/L | |
| Approach | final | final | final | final | PBS |
| D | Yes | No | No | No | Yes |
| D + P + E | Yes | No | Yes | Yes | Yes |
| D + S | Yes | Yes | No | No | Yes |
| D + S + P | Yes | Yes | Yes | No | Yes |

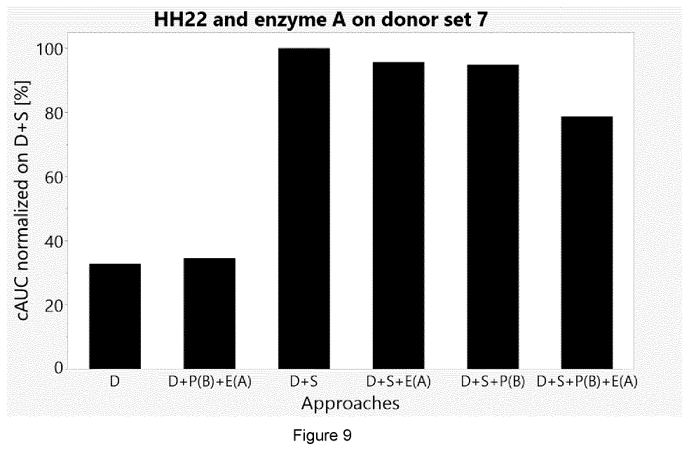

For FIG. 9, the pressure was calculated and represented in a bar chart as cumulated area under the curve (cAUC) in presence of human fecal donor set 7 after 22 h of incubation. Donor (D), Donor+Probiotic+Enzyme (D+P(B)+E(A)), Donor+Substrate (D+S), Donor+Substrate+Enzyme (D+S+E(A)), Donor+Substrate+Probiotic (D+S+P(B)), Donor+Substrate+Probiotic+Enzyme (D+S+P(B)+E(A)). Each column represents the mean of two fecal donors each tested one time.
Results shown in FIG. 3, FIG. 4, FIG. 5, FIG. 6, FIG. 7, and FIG. 9 show that the chosen donor sets had a substantial influence on the possible degree of pressure reduction. Therefore, it is important to use the same donor for each set of comparison.
Results shown in FIG. 9 indicate that HH22 in the approach D+S+P was able to reduce the pressure calculated as cAUC compared to the pressure calculated as cAUC of the D+S approach on different fecal donor sets on average.

### Example 9—Gas Pressure Tests of Bacterial Fructanases and Lactobacillus Strains in Combination

The enzyme E(A) tested in Example 3 and the strain HH22 tested in Example 7 were further tested in combination with each other in a pressure measurement assay with fecal samples. The pressure was continuously measured within the observation period using inhouse developed pressure measuring devices (2ML-FPM).
Human fecal samples were collected, treated and stored as described in Example 3. The Lactobacillus strain HH22 screened in Example 7 is termed as Probiotic (P) in this example.
The approaches Donor (D), Donor+Probiotic+Enzyme (D+P+E), Donor+Substrate (D+S), and Donor+Substrate+Probiotic+Enzyme (D+S+P+E) shown in table 9 were prepared in duplicates of a total of 2 mL per replicate according to the scheme below and incubated at 37° C. within an anaerobic chamber (3% H2, 10% CO2, 87% N2). The pressure of each approach and replicate was measured once every minute over 22 hours using 2ML-FPM devices. For normalization, the cumulative area under the curve (cAUC) was calculated at every measure cycle. The cAUC of approach D+S was set to 100% and the cAUC of all other approaches was set in relation to this.

TABLE 9

| | Fecal | | Strain | Enzyme | |
|---|---|---|---|---|---|
| | sample | Substrate | 1E+8 | 1.5E−2 | |
| | 2.5% | 5 g/L | CFU/mL | g/L | |
| Approach | final | final | final | final | PBS |
| D | Yes | No | No | No | Yes |
| D + P + E | Yes | No | Yes | Yes | Yes |
| D + S | Yes | Yes | No | No | Yes |
| D + S + P + E | Yes | Yes | Yes | Yes | Yes |

Results shown in FIG. 3, FIG. 4, FIG. 5, FIG. 6, FIG. 7, and FIG. 9 show that the chosen donor sets had a substantial influence on the possible degree of pressure reduction. Therefore, it is important to use the same donor for each set of comparison.
Results shown in FIG. 9 show that the combinations of the enzyme A with the strain HH22 in approach D+S+P+E was able to reduce the pressure calculated as cAUC compared to the pressure calculated as cAUC in the corresponding D+S approaches on average.
Results shown in FIG. 9 show that the combinations of the enzymes A, B, C, E or F with the strain HH22 in approach D+S+P+E were able to reduce the pressure calculated as cAUC the most, compared to the pressure calculated as cAUC in the corresponding D+S+P and D+S+E approaches, on average.

### Example 10—Microbiome Analysis

1 ml of the faecal fermentations of donor set 8 was sampled for microbiome analysis at time point 0 hours and at 24 hours and was centrifuged to pellet the microbial cells. Lysis buffer was added directly to the pellet and the sample was transferred to abead tube. DNA extraction was continued following the manufactures protocol for the NucleoSpin soil kit (Machery Nagel). The library preparation and following bioinformatics analysis was conducted as described in “Effect of an enzyme-containing mouthwash on the dental biofilm and salivary microbiome in patients with fixed orthodontic appliances: a randomized placebo-controlled pilot trial”, Hoffstedt et al., 2022. In short, the V3V4 region of the 16S rRNA gene was amplified using universal bacterial primers and the amplicons were sequenced using the MiSeq technology (Illumina) in 2×300 bp paired end mode. The USEARCH pipeline was used to process the data bioinformatically and create an amplicon sequence variant table, where the SILVA SSU 138.1 Ref NR 99 database was used for the taxonomic classification (as described in “The SILVA ribosomal RNA gene database project: improved data processing and web-based tools.” Quast et al., 2013). The table was rarefied to 2283 reads per sample.

### A. Enzyme Induces Growth of Probiotics in Presence of Inulin in Faecal Fermentation

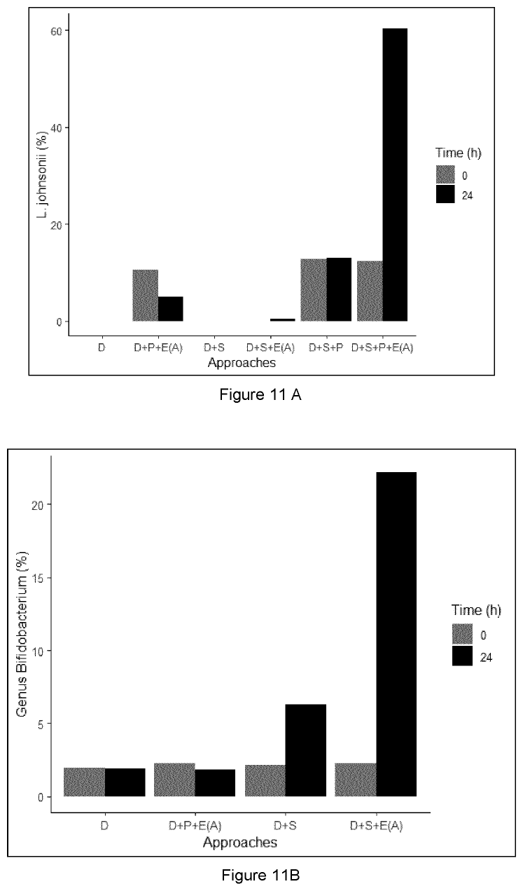

The experimental setup described in Example 3 for donor set 8 was sampled for microbiome analysis. 1 ml sample was taken at time point 0 hours and after 24 hours and prepared for microbiome sequencing. The amplicon sequence variant representing the added probiotic and closely related strains was identified and the average relative abundance across the five donors is represented in FIG. 11A. The different approaches are displayed on the x-axis, Donor (D), Donor+Probiotic+Enzyme (D+P+E(A)), Donor+Substrate (D+S), Donor+Substrate+Enzyme (D+S+E(A)), Donor+Substrate+Probiotic (D+S+P), Donor+Substrate+Probiotic+Enzyme (D+S+P+E(A)). The relative abundance of the probiotic strain was measured for donor set 8, at time point 0 hours (grey bars) and time point 24 hours (black bars). The bar chart shows that at time point 0 hours the probiotic strain was added in similar amounts (10-12%) to approaches D+P+E(A), D+S+P and D+S+P+E(A). For the samples without inulin (D+P+E(A)) the relative abundance of the strain decreases after 24 h, in the samples with inulin and probiotic (D+S+P) no change in abundance is observed but when enzyme is added to the approach (D+S+P+E(A)) the abundance of the probiotic strain increases.

### B. Enzyme Induces Growth of Bifidobdacteria in Presence of Inulin in Faecal Fermentation

The experimental setup described in Example 3 for donor set 8 was sampled for microbiome analysis. 1 ml human fecal sample was taken at time point 0 hours and after 24 hours and prepared for microbiome sequencing. All amplicon sequence variants representing a member of the genus Bifidobacterium were summarized and the average relative abundance across the five donors is represented in FIG. 11B. The different approaches are displayed on the x-axis, Donor (D), Donor+Probiotic+Enzyme (D+P+E(A)), Donor+Substrate (D+S), Donor+Substrate+Enzyme (D+S+E(A)). The relative abundance of Bifidobacterium was measures for five different donors, set 8, at time point 0 hours (grey bars) and time point 24 hours (black bars). The bar chart shows that at time point 0 hours Bifidobacterium takes up approximately 2% of the whole microbial community. This level is stable after 24 hours for samples without inulin added that is approaches (D and D+P+E(A)), the abundance increases slightly when inulin is present that is approach (D+S) and when enzyme is added that is approach (D+S+E(A)), the abundance increases further.

### Deposit of Biological Material

The following biological material has been deposited under the terms of the Budapest Treaty with the Deutsche Sammlung von Mikroorganismen und Zellkulturen GmbH (DSMZ), Mascheroder Weg 1 B, D-38124 Braunschweig, Germany, and given the following accession numbers:

| | Accession | Data of | Origin of |
|---|---|---|---|
| Deposit | No. | Deposit | deposit |
| Lactobacillus | DSM 33901 | 15 Jun. 2021 | Germany |
| johnsonii HH15 | | | |
| Lactobacillus | DSM 34452 | 13 Dec. 2022 | Germany |
| crispatus HH22 | | | |

The strains have been deposited under conditions that assure that access to the culture will be available during the pendency of this patent application to one determined by foreign patent laws to be entitled thereto. The deposit represents substantially pure cultures of the deposited strains. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.
The Lactobacillus HH15 strain was confirmed by 16S, WGS and Biotyping. The Lactobacillus HH22 strain was confirmed by Biotyping.

1-15. (canceled)

16: A composition comprising one or more fructanases and one or more Lactobacillus strains, wherein the composition is capable of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; or
d. any combination thereof.

17: A Lactobacillus strain or a composition thereof, wherein the strain or composition is capable of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; or
d. any combination thereof.

18: A fructanase or a composition wherein the fructanase or composition is capable of:
a. providing relief from or preventing fructan and/or fructose induced abdominal discomfort to a subject;
b. digesting fructan and/or fructose or improving digestion of fructan and/or fructose for a subject;
c. providing digestive comfort after digestion of fructan and/or fructose on consumption of products containing fructan and/or fructose to a subject; or
d. any combination thereof.

19: A Lactobacillus strain, wherein the strain is capable of:
providing relief from or preventing fructan and/or fructose induced abdominal discomfort,
digesting fructan and/or fructose or improving digestion of fructan and/or fructose, and/or
providing digestive comfort after digestion of fructan and/or fructose on consumption of products containing fructan and/or fructose to a subject, and
wherein the Lactobacillus strain in presence of one or more fructanases results in at least 20-50% reduction of gas pressure development when tested against the fecal fermentation of the fiber alone across different donors.

20: A fructanase, wherein the fructanase is capable of:
providing relief from or preventing fructan and/or fructose induced abdominal discomfort,
digesting fructan and/or fructose or improving digestion of fructan and/or fructose, and/or
providing digestive comfort after digestion of fructan and/or fructose on consumption of products containing fructan and/or fructose to a subject, and
wherein the fructanase results in at least 20-50% reduction of gas pressure development, when tested against the fecal fermentation of the fiber alone across different donors.

21: A composition, the composition comprising:
(a) a Lactobacillus strain; and
(b) a fructanase;
wherein the composition is capable of providing relief from or preventing fructan and/or fructose induced abdominal discomfort,
digesting fructan and/or fructose or improving digestion of fructan and/or fructose, and/or
providing digestive comfort after digestion of fructan and/or fructose on consumption of products containing fructan and/or fructose to a subject.

22: The composition according to claim 21, wherein the Lactobacillus strain is Lactobacillus johnsonii or Lactobacillus crispatus.

23: The composition according to claim 21, wherein the Lactobacillus strain is selected from the group consisting of:
a. Lactobacillus johnsonii strain HH15 deposited under deposit accession number DSM 33901, or the Lactobacillus strain having all the characteristics of Lactobacillus johnsonii strain deposited under deposit accession number DSM 33901, or a mutant of Lactobacillus johnsonii strain deposited under deposit accession number DSM 33901, and
b. Lactobacillus crispatus HH22 strain deposited under deposit accession number DSM 34452, or the Lactobacillus strain having all the characteristics of Lactobacillus crispatus strain deposited under deposit accession number DSM 34452, or a mutant of Lactobacillus crispatus strain deposited under deposit accession number DSM 34452.

24: The composition according to claim 21, wherein the fructanase is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).

25: The composition according to claim 24, wherein the exo-inulinase is a polypeptide selected from the group consisting of:
a. a polypeptide having at least 80% sequence identity to SEQ ID NO: 1,
b. a polypeptide derived from SEQ ID NO: 1 by having 1-30 alterations,
c. a polypeptide derived from the polypeptide of (a) or (b), wherein the N- and/or C-terminal end has been extended by addition of one or more amino acids, and
d. a fragment of the polypeptide of (a), (b), or (c).

26: The composition according to claim 24, wherein the endo-inulinase is a polypeptide selected from the group consisting of:
a. a polypeptide having at least 80% sequence identity to SEQ ID NO: 2;
b. a polypeptide derived from SEQ ID NO: 2 by having 1-30 alterations,
c. a polypeptide derived from the polypeptide of (a) or (b), wherein the N- and/or C-terminal end has been extended by addition of one or more amino acids, and
d. a fragment of the polypeptide of (a), (b), or (c).

27: The composition according to claim 24, wherein the invertase is a polypeptide selected from the group consisting of:
a. a polypeptide having at least 80% sequence identity to SEQ ID NO: 3;
b. a polypeptide derived from SEQ ID NO: 3 by having 1-30 alterations,
c. a polypeptide derived from the polypeptide of (a) or (b), wherein the N- and/or C-terminal end has been extended by addition of one or more amino acids, and
d. a fragment of the polypeptide of (a), (b), or (c).

28: A method for providing relief from or preventing fructan and/or fructose induced abdominal discomfort, digesting fructan and/or fructose or improving digestion of fructan and/or fructose, and providing digestive comfort after digestion of fructan and/or fructose on consumption of products containing fructan and/or fructose to a subject; the method comprising providing to a subject in need thereof an effective amount a composition comprising one or more fructanases and one or more Lactobacillus strains.

29: The method according to claim 28, wherein the Lactobacillus strain is Lactobacillus johnsonii or Lactobacillus crispatus.

30: The method according to claim 28, wherein the one or more fructanases is selected from the group consisting of: endo-inulinase (EC 3.2.1.7), exo-inulinase (EC 3.2.1.80), beta-fructofuranosidase, invertase (EC 3.2.1.26) and levanase (EC 3.2.1.65).


SEQUENCE LISTING (3 sequences)

SEQ ID NO: 1 - protein, 518 aa, Aspergillus Niger
FNYDQPYRGQYHFSPQKNWMNDPNGLLYHNGTYHLFFQYNPGGIEWGNISWGHATSEDLTHWEEQPVALLARGYGSDVTEMYFSGSAVADVNNTSGFGKDGKTPLVAMYTSYYPVAQTLPSGQTVQEDQQSQSIAYSLDDGLTWTTYDAANPVIPNPPQPYQAQYQNFRDPFVFWHDESHKWVVVTSIAELHKLAIYTSDNLKDWKLVSEFGPYNAQGGVWECPGLFKLPLDGGSSTKWVITSGLNPGGPPGTVGSGTQYFVGEFDGTTFTPDADTVYPGNSTANWMDWGPDFYAAAGYNGLSIKDHVHIGWMNNWQYGANIPTYPWRSAMAIPRHLALKTINNKTTLVQQPQEAWSSISSKHPLYSRTYSTFSEGSTNASTTGETFRVDLSFSATSKASTFAIALRASANFTEQTLAGYDFAKQQIFLDRTKSGDVSFDNTFASVYHGPLVPDSTGMVRLSIFVDRSSVEVFGGQGETTLTAQIFPSNDAVHARLVSTGGATEDVRVDVHNITSTWN

SEQ ID NO: 2 - protein, 491 aa, Aspergillus Niger
NDYRPSYHFTPDQYWMNEPNGLIKIGSTWHLFFQHNPTANVWGNICWGHATSTDLMHWAHKPTAIADENGVEAFTGTAYYDPNNTSGLGDSANPPYLAWFTGYTTSSQTQDQRLAFSVDNGATWTKFQGNPIISTSQEAPHDITGGLESRDPKVFFHRQSGNWIMVLAHGGQDKLSFWTSADTINWTWQSDLKSTSINGLSSDITGWEVPDMFELPVEGTEETTWVVMMTPAEGSPAGGNGVLAITGSFDGKSFTADPVDASTMWLDNGRDFDGALSWVNVPASDGRRIIAAVMNSYGSNPPTTTWKGMLSFPRTLSLKKVGTQQHFVQQPITELDTISTSLQILANQTITPGQTLLSSIRGTALDVRVAFYPDAGSVLSLAVRKGASEQTVIKYTQSDATLSVDRTESGDISYDPAAGGVHTAKLEEDGTGLVSIRVLVDTCSVEVFGGQGEAVISDLIFPSDSSDGLALEVTGGNAVLQSVDVRSVSLE

SEQ ID NO: 3 - protein, 613 aa, Aspergillus Niger
ASPSMQTRASVVIDYNVAPPNLSTLPNGSLFETWRPRAHVLPPNGQIGDPCLHYTDPSTGLFHVGFLHDGSGLSSATTDDLATYKDLNQGNQVIVPGGINDPVAVFDGSVIPSGINGLPTLLYTSVSYLPIHWSIPYTRGSETQSLAVSSDGGSNFTKLDQGPVIPGPPFAYNVTAFRDPYVFQNPTLDSLLHSKNNTWYTVISGGLHGKGPAQFLYRQYDPDFQYWEFLGQWWHEPTNSTWGNGTWAGRWAFNFETGNVFSLDEYGYNPHGQIFSTIGTEGSDQPVVPQLTSIHDMLWVSGNVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSELESTTVYYQFSNESIIVDRSNTSAAARTTDGIDSSAEAGKLRLFDVLNDGEQAIETLDLTLVVDNSVLEIYANGRFALSTWVRSWYANSTNISFFQNGVGGVAFSNVTVSEGLYDAWPDRQS